(19) United States
(12) Patent Application Publication
(10) Pub. No.: US 2013/0136693 A1
(43) Pub. Date: May 30, 2013

(54) Crystal Forms of 2--Adenosine

(71) Applicants: Allan R. Moorman, Durham, NC (US); Michael H. O'Neal, Painesville, OH (US)

(72) Inventors: Allan R. Moorman, Durham, NC (US); Michael H. O'Neal, Painesville, OH (US)

(73) Assignee: King Pharmaceuticals Research and Development, Inc, Madison, NJ (US)

(21) Appl. No.: 13/546,595

(22) Filed: Jul. 11, 2012

Related U.S. Application Data
(63) Continuation of application No. 12/918,213, filed on Nov. 3, 2010, now abandoned.
(60) Provisional application No. 61/032,561, filed on Feb. 29, 2008.
(60) Provisional application No. 61/038,251, filed on Mar. 20, 2008.

(51) Int. Cl. C07H 19/167; A61K 49/00; A61K 31/7076

(52) U.S. Cl. CPC C07H 19/167; A61K 31/7076; A61K 49/00
USPC 424/9.1; 536/27.61; 514/46

(57) ABSTRACT

The present invention provides novel crystalline polymorphic forms of 2-cyclohexylmethylidenehydrazino adenosine, also known as binodenoson, methods of making the same, and methods for the manufacture of a pharmaceutical composition by employing such crystal forms, in particular, for the use of binodenoson in a subject, in need thereof, as a pharmacological stress agent to produce coronary vasodilation.

### FIELD OF INVENTION

The present invention provides crystal forms of 2-{2-[(cyclohexyl)methylene]-hydrazino}adenosine, also known as binodenoson, methods of making the same, and methods for the manufacture of a pharmaceutical composition by employing such crystal forms, in particular, for the use of binodenoson in a subject, in need thereof, as a pharmacological stress agent to produce coronary vasodilation.

### BACKGROUND OF THE INVENTION

Adenosine has been known since the early 1920's to have potent vasodilator activity. It is a local hormone released from most tissues in the body during stress, especially hypoxic and ischemic stress (Olsson et al., Physiological Reviews, 70(3), 761-845, 1990). As such, adenosine and adenosine uptake inhibitors are now commonly used to simulate the stress condition for diagnostic purposes in subjects who cannot exercise adequately to produce a diagnostic exercise stress study (The Medical Letter, 33(853), 1991).

Thallium-201 myocardial perfusion imaging is currently the most common approach in the use of stress-simulating agents as a means of imaging the coronary vessels to obtain a diagnosis of coronary artery disease. This is effected by injection of the stress agent such as adenosine at a dose of about 1 mg/kg body weight, followed by injection of the radionuclide, thallium-201, and scanning with a rotating gamma counter to image the heart and generate a scintigraph (McNulty, Cardiovascular Nursing, 28(4), 24-29, 1992).

The use of adenosine and like-acting analogs is associated with certain side-effects. Adenosine acts on at least two subclasses of adenosine receptors, A₁ or A₂, both of which are found in the heart. The A₁ receptor subtype, when activated by adenosine, among other actions, slows the frequency and conduction velocity of the electrical activity that initiates the heart beat. Sometimes adenosine, particularly at doses near 1 mg/kg, even blocks (stops) the heart beat during this diagnostic procedure, a highly undesirable action. The A₂ receptor subtype is found in blood vessels and is further divided into A_{2A} and A_{2B} receptor subtypes (Martin et al., Journal of Pharmacology and Experimental Therapeutics, 265(1), 248-253, 1993). It is the A_{2A} receptor that is specifically responsible for mediating coronary vasodilation, the desired action of adenosine in the diagnostic procedure. Thus, the side-effects of adenosine and adenosine releasing agents result substantially from non-selective stimulation of the various adenosine receptor subtypes. Clearly, a better procedure would be to use a substance as a stress agent that selectively activates only the A_{2A} receptor, is short acting and works at doses substantially below 1 mg/kg body weight.

Binodenoson is a highly selective adenosine A_{2A} receptor agonist that has relatively lower affinity for the adenosine A₁, A_{2B} and A₃ receptor subtypes and, thus, has a therapeutic utility as a pharmacological stress agent to produce coronary vasodilation. In addition to its potential diagnostic applications, binodenoson may also be useful for treating certain respiratory disorders such as asthma, chronic obstructive pulmonary disease (COPD), and other obstructive airway diseases exacerbated by heightened bronchial reflexes, inflammation, bronchial hyper-reactivity and bronchospasm.

Binodenoson and its preparation are disclosed in U.S. Pat. No. 5,278,150 and by Niiya, R. in J. Med. Chem., 35, 4557-4561, 1992.

### SUMMARY OF THE INVENTION

The present invention provides crystal forms of binodenoson of the formula

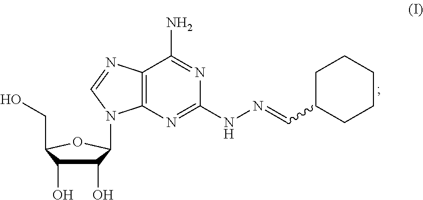

methods of making the same, and methods for the manufacture of a pharmaceutical composition by employing such crystal forms, in particular, for the use of binodenoson in a subject, in need thereof, as a pharmacological stress agent to produce coronary vasodilation. The crystal forms of the present invention are especially useful in the manufacture of pharmaceutical compositions for achieving coronary vasodilation in subjects who cannot exercise adequately.

Pharmaceuticals that exhibit polymorphism offer unique challenges in product development. Thus, it is essential to understand the polymorphic behavior of crystalline solids and their relative thermodynamic stability to avoid complications during processing and development. Conversion of one crystal form into unknown amounts of different crystalline or amorphous forms during processing or storage is undesirable, and in many cases would be regarded as analogous to the appearance of unquantified amounts of impurities in the product. Therefore, it is generally desirable to manufacture the drug substance in the most stable solid state form, thereby minimizing the possibility of less stable forms being generated during storage. However, the less stable solid state forms (polymorphs) may offer advantages over the most stable form, such as enhanced solubility, reduced hygroscopicity, and improved bulk properties e.g., improved flow properties and bulk density, any of which may make them more desirable than the most stable solid state form. These differences in physicochemical properties among the polymorphs of a drug substance are well known to those skilled in the art, and have been discussed widely in the literature (See for example “Polymorphism in Pharmaceutical Solids”, edited by Harry G. Brittain. Vol. 95, Drugs and the Pharmaceutical Sciences, Marcel Dekker, Inc. 270 Madison Avenue, New York, N.Y. 10016. Copyright 1999).

Accordingly, there is a need to characterize different solid forms of a drug substance, e.g., crystalline forms of binodenoson, which are stable and have good bulk properties and are easy to manage in the drying or grinding processes following the final stage of the chemical synthesis of the drug substance. The crystal forms of the present invention, in particular, the crystal form designated herein as the Form II crystal form, exhibits the desired improved properties as described herein.

Other objects, features, advantages and aspects of the present invention will become apparent to those skilled in the art from the following description, appended claims and accompanying drawings. It should be understood, however, that the following description, appended claims, drawings and specific examples, while indicating preferred embodiments of the present invention, are given by way of illustration only. Various changes and modifications within the spirit and scope of the disclosed invention will become readily apparent to those skilled in the art from reading the following.

### BRIEF DESCRIPTION OF THE DRAWINGS

FIG. 1A shows an overlay of differential scanning calorimetry and thermogravimetric data of Form I crystals of binodenoson containing about 0.5 wt-% of water and about 4 wt-% of ethanol.

FIG. 1B shows differential scanning calorimetry data of Form I crystals of binodenoson in an anhydrous form.

FIG. 2 shows a X-ray powder diffraction diagram of Form I crystals of binodenoson.

FIG. 3 shows an infrared reflectance spectrum of Form I crystals of binodenoson.

FIG. 4 shows a Raman spectrum of Form I crystals of binodenoson.

FIG. 5 shows an overlay of differential scanning calorimetry and thermogravimetric data of Form II crystals of binodenoson.

FIG. 6A shows a thermal ellipsoid diagram of Form II hydrate of binodenoson.

FIG. 6B shows a X-ray powder diffraction diagram of Form II crystals of binodenoson.

FIG. 7 shows an infrared reflectance spectrum of Form II crystals of binodenoson.

FIG. 8 shows a Raman spectrum of Form II crystals of binodenoson.

FIG. 9 shows an overlay of differential scanning calorimetry and thermogravimetric data of Form III crystals of binodenoson.

FIG. 10 shows a X-ray powder diffraction diagram of Form III crystals of binodenoson.

FIG. 11 shows an infrared reflectance spectrum of Form III crystals of binodenoson.

FIG. 12 shows a Raman spectrum of Form III crystals of binodenoson.

FIG. 13 shows an overlay of differential scanning calorimetry and thermogravimetric data of Form IV crystals of binodenoson.

FIG. 14 shows a X-ray powder diffraction diagram of Form IV crystals of binodenoson.

FIG. 15 shows an infrared reflectance spectrum of Form IV crystals of binodenoson.

FIG. 16 shows a Raman spectrum of Form IV crystals of binodenoson.

FIG. 17 shows an overlay of differential scanning calorimetry and thermogravimetric data of Form V crystals of binodenoson.

FIG. 18 shows a X-ray powder diffraction diagram of Form V crystals of binodenoson.

FIG. 19 shows an infrared reflectance spectrum of Form V crystals of binodenoson.

FIG. 20 shows a Raman spectrum of Form V crystals of binodenoson.

FIG. 21 shows an overlay of differential scanning calorimetry and thermogravimetric data of Form VI crystals of binodenoson.

FIG. 22 shows a X-ray powder diffraction diagram of Form VI crystals of binodenoson.

FIG. 23 shows an infrared reflectance spectrum of Form VI crystals of binodenoson.

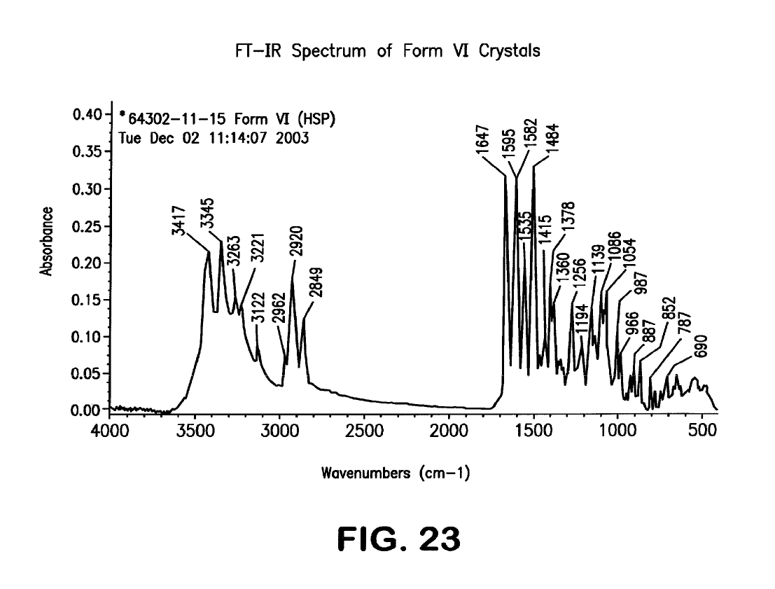

FIG. 24 shows a Raman spectrum of Form VI crystals of binodenoson.

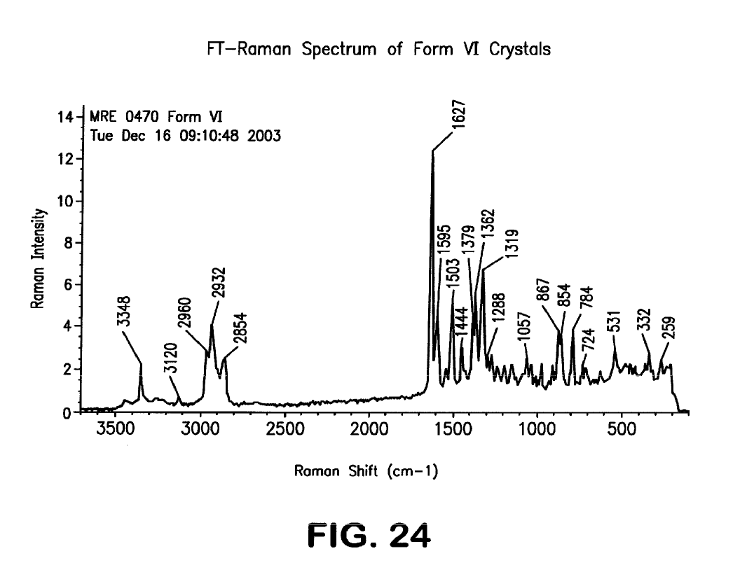

FIG. 25 shows a differential scanning calorimetry data for amorphous binodenoson.

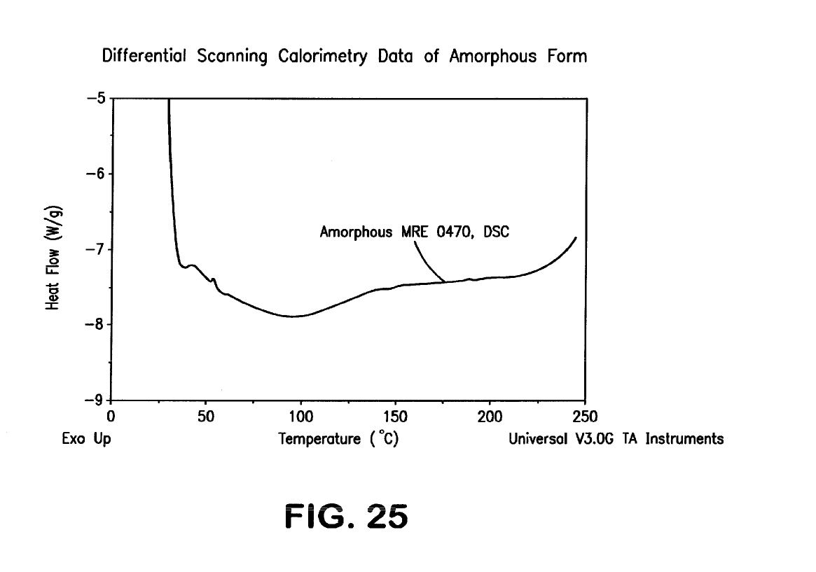

FIG. 26 shows a X-ray powder diffraction diagram of amorphous binodenoson.

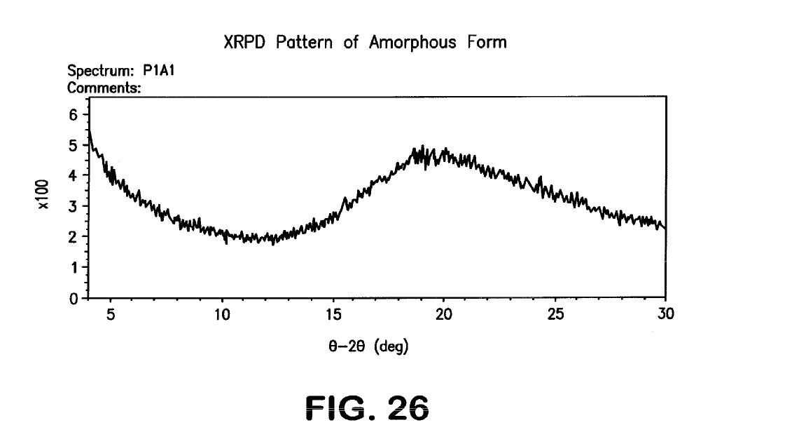

FIG. 27 shows a Raman spectrum of amorphous binodenoson.

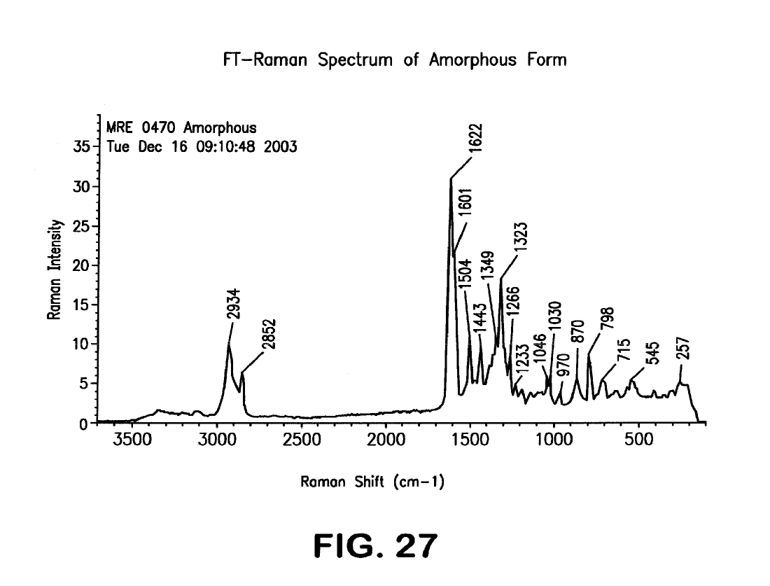

FIG. 28 shows an overlay plot of X-ray powder diffraction diagrams of Form V crystals with the amorphous form of binodenoson. Form V crystal form often develops slowly, and may take many days to develop under many slurry conditions: bottom pattern, amorphous form; top pattern, Form V after 15 days.

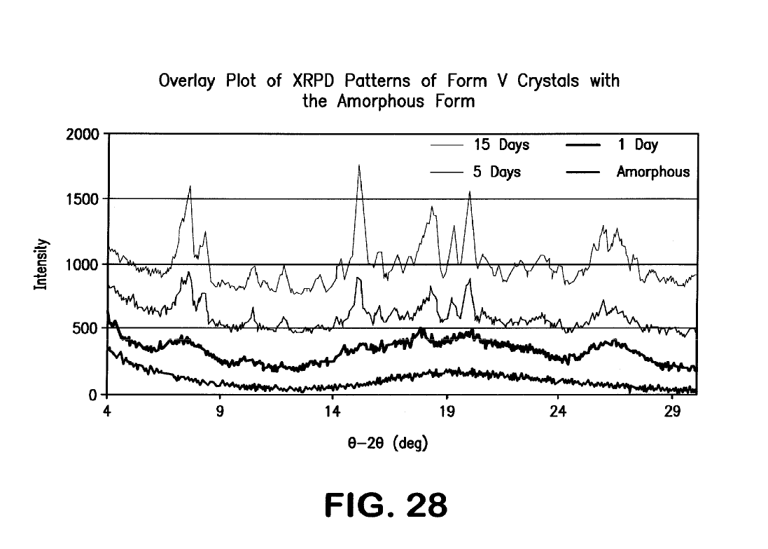

### DETAILED DESCRIPTION OF THE INVENTION

As described above, the present invention provides crystal forms of binodenoson of the formula

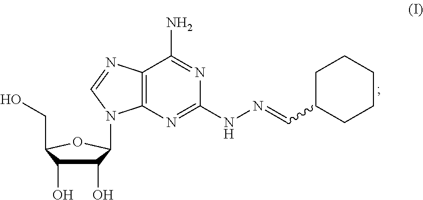

and methods of making the same. The crystal forms of binodenoson may be employed for the manufacture of a pharmaceutical composition comprising an effective amount of binodenoson for the use of binodenoson in a subject as a pharmacological stress agent to produce coronary vasodilation. The crystal forms of the present invention are especially useful in the manufacture of pharmaceutical compositions for achieving coronary vasodilation in subjects who cannot exercise adequately.

As employed throughout the description and appended claims, the term “crystals” or “crystal forms” of the present invention refers to, as appropriate, crystal forms of binodenoson, designated as Form I, Form II, Form III, Form IV, Form V and Form VI, as defined herein below, and are substantially free of all other alternative crystalline and amorphous forms.

The term “substantially free” when referring to a designated crystal form of binodenoson means that the designated crystal form contains less than 20% (by weight) of any alternate polymorphic form(s) of binodenoson, preferably less than 10% (by weight) of any alternate polymorphic form(s) of binodenoson, more preferably less than 5% (by weight) of any alternate polymorphic form(s) of binodenoson, and most preferably less than 3% (by weight) of any alternate polymorphic forms of binodenoson.

The crystal forms of the present invention may be characterized by measuring at least one of the following physico-chemical properties: 1) a melting point (m.p.) and/or thermal differential scanning calorimetry (DSC) data; 2) a X-ray powder diffraction pattern; 3) an infrared reflectance spectrum; and/or 4) a Raman spectrum.

The melting points and/or thermal DSC data may be measured, e.g., using a TA Instruments differential scanning calorimeter 2910 (DSC method). The sample is placed into an aluminium DSC pan, and the weight is recorded. The pan is covered with a lid and then crimped or hermetically sealed. Each sample is heated under nitrogen purge at a rate of 1-50° C./min. Indium metal is used as the calibration standard. Reported temperatures are at the transition onset.

In addition to thermal DSC data, thermogravimetric analyses (TGA) may be performed, e.g., using a TA Instrument's 2950 thermogravimetric analyzer. Each sample is placed in an aluminium sample pan and inserted into the TG furnace. Samples are heated under nitrogen at a rate of 1-50° C./min. Nickel and Alumel™ are used as the calibration standards.

X-ray powder diffraction (XRPD) analyses may be performed, e.g., using a Philips 3100X-ray powder diffractometer equipped with a fine focus X-ray tube using Cu radiation at 1.54 Å. The system includes a Philips Norelco wide angle goniometer and a Theta XRD automation system. The voltage and amperage of the X-ray generator are set at 40 kV and 20 mA, respectively. The radiation is monochromatized by a graphite crystal. The scan range is 4-30 °2θ and the step size is 0.05 °2θ (count time per step=2 sec). The slits are fixed at 1° divergence/0.2° receiving. Data are collected at ambient temperature. Powder samples (approximately 0.3 g) are mounted on a low background glass plate using a top mounting approach. Samples are analyzed with and without grinding, and as demonstrated that grinding the samples before analysis does not change the crystalline form.

Infrared reflectance spectra may be acquired on a Fourier transform infrared (FT-IR) spectrophotometer (Nicolet Model 510M) equipped with a Harrick internal reflection nanosampler accessory (HSP). A small amount of the sample is placed on the surface of the reflectance attachment and approximately 2-4 lb of pressure is applied to enhance sample contact with the instrument optics. The infrared spectra are obtained over the region of 4000 to 400 cm⁻¹.

FT-Raman spectra may be acquired, e.g., on a FT-Raman 960 spectrometer (Thermo Nicolet) configured for backscattering. This spectrometer uses an excitation wavelength of 1064 nm. Approximately 1 W of Nd:YVO₄ laser power is used to irradiate the sample. The Raman spectra are measured using the 180 degree back scattering sampling geometry. The samples are prepared for analysis by placing the material in a sealed glass NMR tube and placed into the sampling geometry. The sample focus is optimized for the maximum Raman intensity, and a total of 64 sample scans are collected at a spectral resolution of 4 cm⁻¹. The Raman spectra are obtained over the spectral range of 3700 to 100 cm⁻¹ (Strokes).

One of ordinary skill in the art will appreciate that the physico-chemical properties discussed herein above may be obtained with a measurement error that is dependent upon the measurement conditions employed. In particular, it is generally known that intensities in an X-ray diffraction pattern may fluctuate depending upon measurement conditions employed. It should be further understood that relative intensities may also vary depending upon experimental conditions and, accordingly, the exact order of intensity should not be taken into account. Additionally, a measurement error of diffraction angle for a conventional X-ray diffraction pattern is typically about 5% or less, e.g., ±0.2 °2θ, and such degree of measurement error should be taken into account as pertaining to the aforementioned diffraction angles. Consequently, it is to be understood that the crystal forms of the instant invention are not limited to the crystal forms that provide X-ray diffraction patterns completely identical to the X-ray diffraction patterns depicted in the accompanying Figures disclosed herein. Any crystal forms that provide X-ray diffraction patterns substantially identical to those disclosed in the accompanying Figures fall within the scope of the present invention. The ability to ascertain substantial identities of X-ray diffraction patterns is within the purview of one of ordinary skill in the art. A discussion of the theory of powder X-ray diffraction patterns can be found, e.g., in “X-Ray Diffraction Procedures” by Klug and Alexander, J. Wiley, New York (1974).

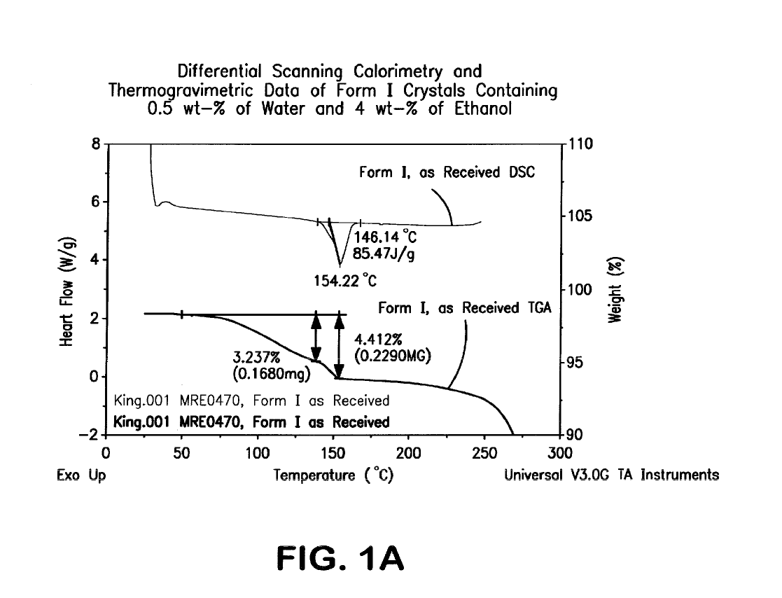

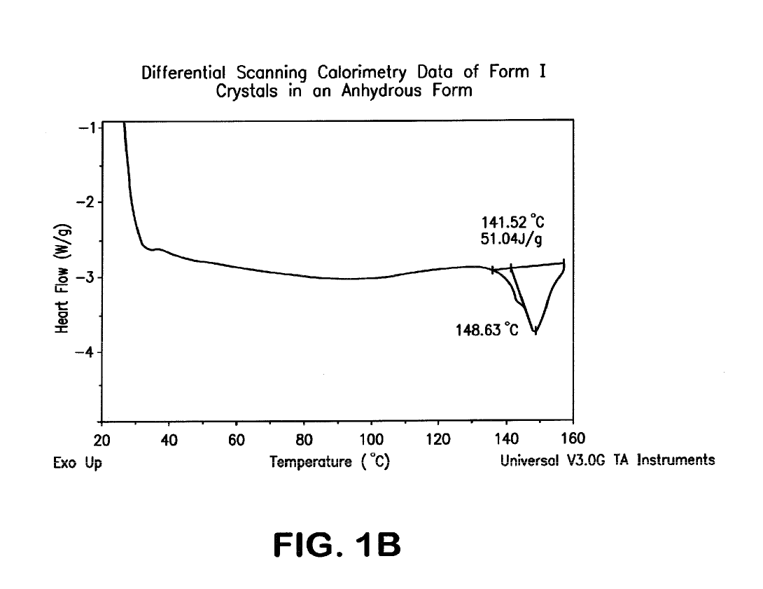

In one aspect, the present invention provides a crystal form of binodenoson, designated herein as Form I, that is characterized by thermal DSC data, as measured by the DSC method described herein above, substantially identical to those depicted in FIG. 1A or FIG. 1B.

Form I crystal form of binodenoson may contain residual solvent(s), or it may be in an anhydrous form, and may be obtained by a variety of techniques, e.g., by slow crystallization from lower alcohols such as methanol (MeOH) and ethanol (EtOH) under anhydrous conditions.

For example, KF analysis performed using an oven attachment at 125° C. under nitrogen atmosphere indicates that Form I crystals may contain about 0.5% of water by weight (wt-%). Furthermore, TGA and ¹H-NMR indicate that Form I crystals may also contain approximately 4 wt-% of ethanol (FIG. 1A).

In accordance with the TG data, after a few minutes at 125° C. in the KF oven, most of the weight loss from ethanol and water is complete affording Form I crystals in an anhydrous form (FIG. 1B). The crystalline form does not change significantly upon drying under nitrogen as determined by XRPD. A comparison of the calorimetric behavior of the anhydrous Form I crystals and the Form I crystals containing residual solvents (“undried”) is summarized in Table 1:

TABLE 1

| Crystalline | DSC Heating | Extrapolated | Heat of Transition |
|---|---|---|---|
| Form | Rate (° C./min) | Onset Temp. (° C.) | (J/g) |
| Anhydrous | 1 | 133 | 23 |
| Form I | 10 | 139 | 41 |
| | 50 | 146 | 26 |
| Undried | 1 | 133 | 48 |
| Form I | 10 | 146 | 85 |
| | 50 | 158 | 101 |

Thermal DSC data of Form I crystals of binodenoson exhibit a single endotherm with an extrapolated onset melting temperature in the range of about 139° C. (anhydrous) to about 146° C. (undried) when heated at 10° C./min. The undried Form I crystals exhibit a larger heat of transition than the anhydrous Form I crystals at all heating rates. The endothermic event observed with the anhydrous Form I crystals is attributed to melting. The endothermic event observed with the undried Form I crystals is attributed to the heat of vaporization (due to the presence of residual solvents) in addition to the heat attributable to melting. The undried Form I crystals exhibit an increase in the heat of transition with increasing heating rate. This is attributable to the removal of more residual solvent (before melting) when using a low heating rate relative to removal of less residual solvent (before melting) when using a higher heating rate. The removal of residual solvents occurs over a broad temperature range and does not give a distinct or characteristic DSC endotherm.

As already discussed, the crystalline structure of Form I does not change significantly upon removal of the residual solvent(s). The thermal DSC and TGA data indicate that the residual solvent(s) in the undried Form I crystal form is not released in an abrupt thermal event generally characteristic of solvates. Yet, the solvent(s) does not seem to be entirely removed even when the sample is heated above the boiling point of the solvent. This behavior is more consistent with a channel solvate than a true, stoichiometric solvate.

A moisture sorption analysis of anhydrous Form I crystals of binodenoson show a weight gain of approximately 1.3% when exposed to a relative humidity of about 94% over a period of 14 days at 25° C. indicating that Form I is non-hygroscopic. The crystal form remains the same after moisture sorption analysis.

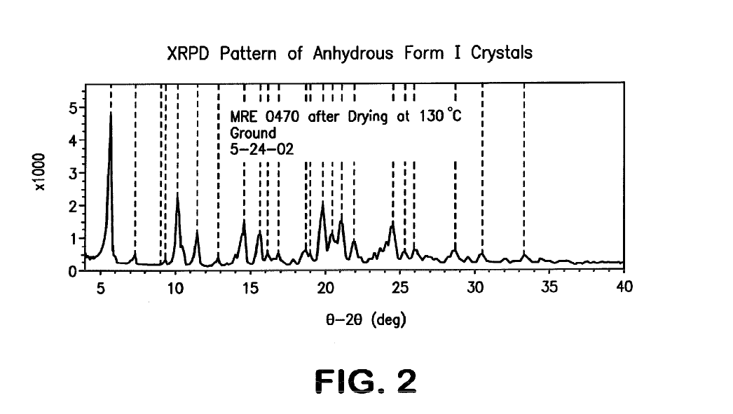

An example of an X-ray diffraction pattern exhibited by Form I crystal form is substantially identical to that depicted in FIG. 2, having characteristic peaks, expressed in degrees 2-theta (20), of about 5.7±0.2, 10.2±0.2, 14.6±0.2, 19.9±0.2, 21.1±0.2 and 24.6±0.2. The present invention also provides a Form I crystal form that exhibits a X-ray diffraction pattern substantially the same as that depicted in FIG. 2, having characteristic diffraction peaks expressed in degrees 2-theta, and relative intensities (I/I₁) of approximately the values shown in Table 2 herein below:

TABLE 2
Form I crystals of binodenoson

| Angle (deg 2θ) | Relative intensity (I/I₁) |
|---|---|
| 5.7 ± 0.2 | 100 |
| 10.2 ± 0.2 | 40 |
| 11.4 ± 0.2 | 22 |
| 14.4 ± 0.2 | 21 |
| 14.6 ± 0.2 | 25 |
| 15.6 ± 0.2 | 21 |
| 19.9 ± 0.2 | 38 |
| 20.5 ± 0.2 | 21 |
| 20.8 ± 0.2 | 17 |
| 21.1 ± 0.2 | 29 |
| 22.0 ± 0.2 | 17 |
| 24.2 ± 0.2 | 16 |
| 24.6 ± 0.2 | 27 |

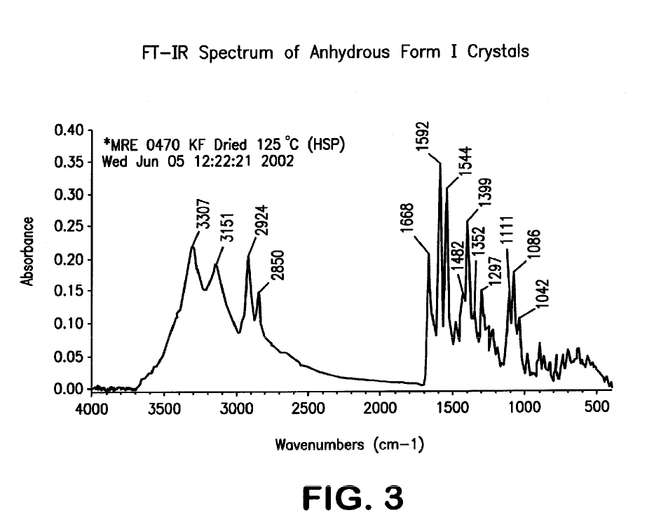

An example of an infrared reflectance spectrum of Form I crystals obtained by the diffuse reflectance method is shown in FIG. 3, and is characterized by reflectance bands at about 1668±2 and 1593±2 cm⁻¹.

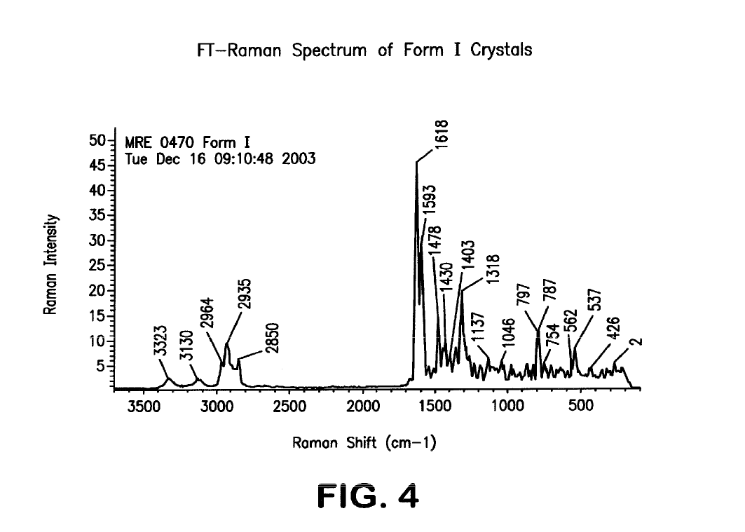

An example of a FT-Raman spectrum of a Form I crystals obtained by the method described herein above is shown in FIG. 4, and is characterized by Raman shifts at about 1618±2 and 1593±2 cm⁻¹.

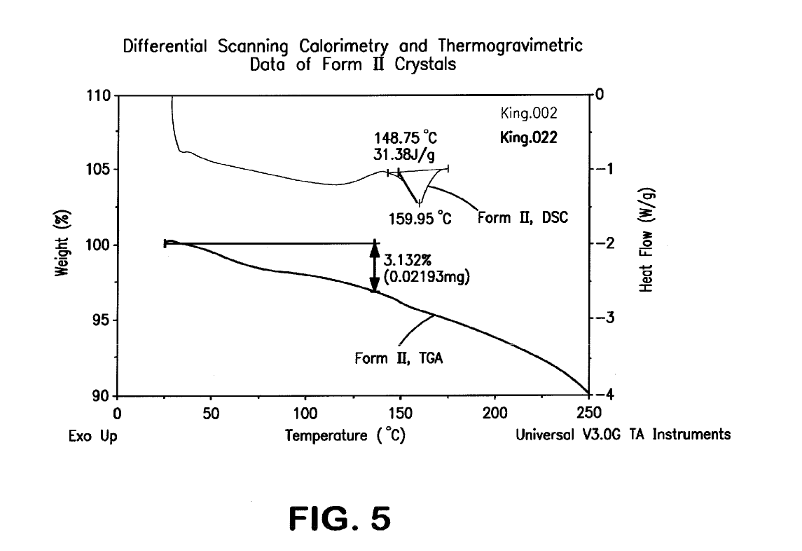

In another aspect, the present invention provides a crystal form of binodenoson, designated herein as Form II, that is characterized by thermal DSC data, as measured by the DSC method described herein above, substantially identical to those depicted in FIG. 5.

Form II crystal form may be obtained in a hydrated form or in an anhydrous form. For example, slow crystallization from lower alcohols such as MeOH and EtOH in the presence of residual water provides hydrated Form II crystals. Interestingly, drying the hydrated Form II crystals does not change the XRPD pattern, indicating that the hydrate is isostructural with the anhydrous crystalline form.

An example of thermal DSC data of Form II crystals of binodenoson exhibit a single endotherm with an extrapolated onset melting temperature in the range of about 149° C. to about 154° C. when heated at 10° C./min.

TG analysis of Form II crystals indicates that samples of Form II crystals often lose approximately 2% of weight over a temperature range of 25° C. to 50° C.

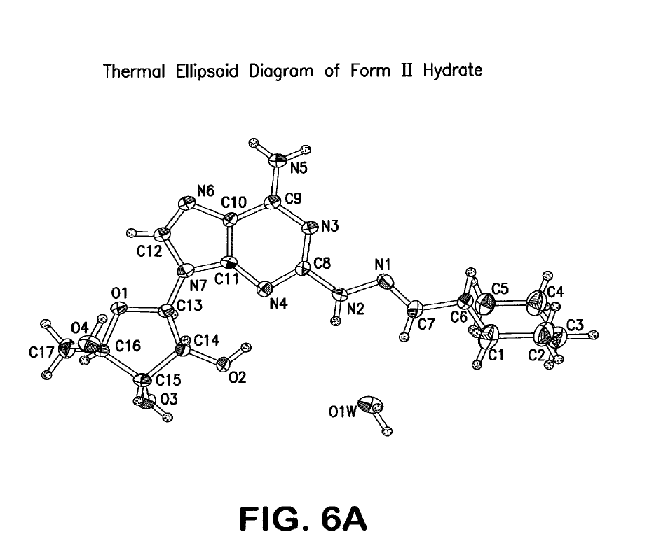

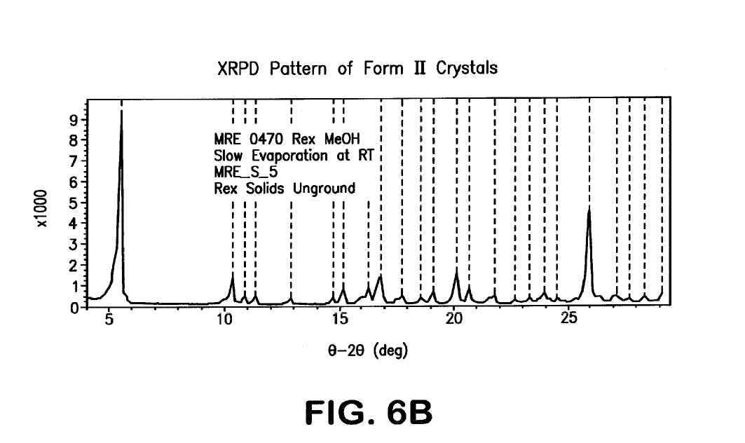

A single crystal structure of Form II crystal form has been obtained and found to be orthorhombic within the P2(1)2(1)2(1) space group having a unit cell with: a=6.8331(17) Å (α=90°), b=8.801(2) Å (β=90°), and c=32.861(8) Å (γ=90°). The structural solution indicated that the material was in a monohydrate form, and that the stereochemistry of the hydrazone double bond is in the E-configuration. Furthermore, examination of the structure indicated that channels exist in the lattice structure which may enable facile sorption and/or desorption of small solvent molecules without much change in the structure. The predicted XRPD pattern from the structural solution is very similar to the empirically observed pattern. A thermal ellipsoid plot of the molecular configuration of Form II hydrate is shown in FIG. 6A.

An example of an X-ray diffraction pattern exhibited by a Form II crystal form (anhydrous or hydrated) is substantially identical to that depicted in FIG. 6B, having characteristic peaks, expressed in degrees 2-theta (26), of about 5.5±0.2, 10.4±0.2, 16.8±0.2, 20.2±0.2 and 26.0±0.2. The present invention also provides a Form II crystal form that exhibits a X-ray diffraction pattern substantially the same as that depicted in FIG. 6B, having characteristic diffraction peaks expressed in degrees 2-theta, and relative intensities (I/I₁) of approximately the values shown in Table 3 herein below:

TABLE 3
Form II crystals of binodenoson

| Angle (deg 2θ) | Relative intensity (I/I₁) |
|---|---|
| 5.5 ± 0.2 | 100 |
| 10.4 ± 0.2 | 15 |
| 16.8 ± 0.2 | 15 |
| 20.2 ± 0.2 | 18 |
| 26.0 ± 0.2 | 50 |

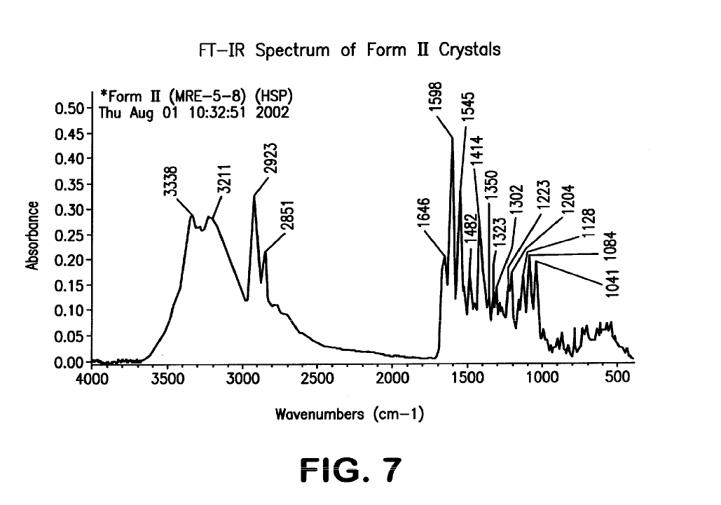

An example of an infrared reflectance spectrum of a Form II crystal form obtained by the diffuse reflectance method is shown in FIG. 7, and is characterized by reflectance bands at about 1646±2 and 1598±2 cm⁻¹.

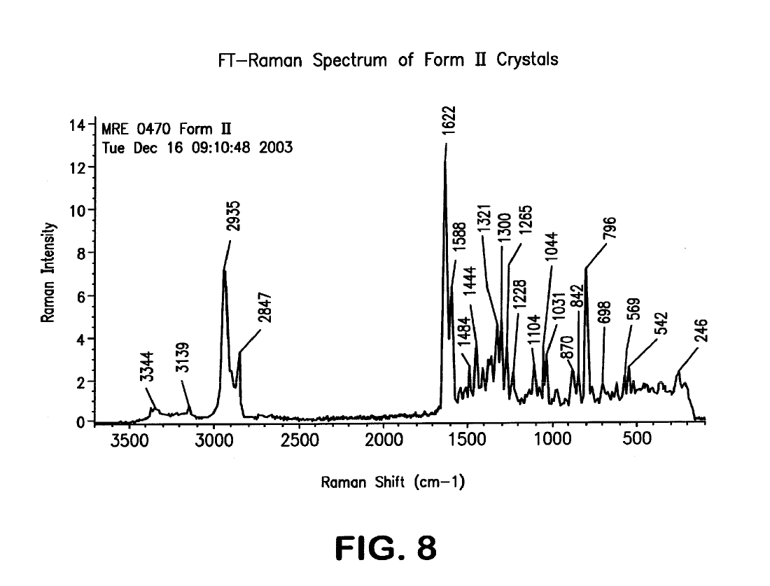

An example of a FT-Raman spectrum of a Form II crystal form obtained by the method described herein above is shown in FIG. 8, and is characterized by Raman shifts at about 1622±2 and 1588±2 cm⁻¹.

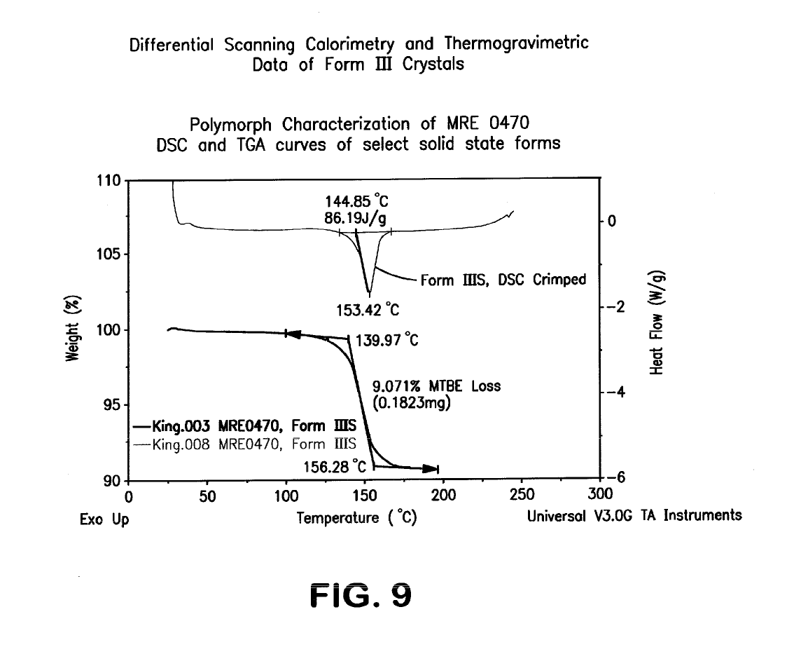

In yet another aspect, the present invention provides a crystal form of binodenoson, designated herein as Form III, that is characterized by thermal DSC data, as measured by the DSC method described herein above, substantially identical to those depicted in FIG. 9.

Form III crystals may be obtained, e.g., by crystallization of binodenoson, in any of its forms, from neat methyl t-butyl ether (MTBE).

Thermal DSC data of Form III crystals of binodenoson exhibit a single endotherm with an extrapolated onset melting temperature in the range of about 142° C. to about 145° C. when heated at 10° C./min.

TGA of Form III crystal form show a characteristic weight loss of approximately 9% between 100° C. and 195° C. corresponding to loss of solvated MTBE. The ¹H-NMR spectrum of the Form III crystals confirms the presence of MTBE. The Form III crystal form appears to be a MTBE hemi-solvate.

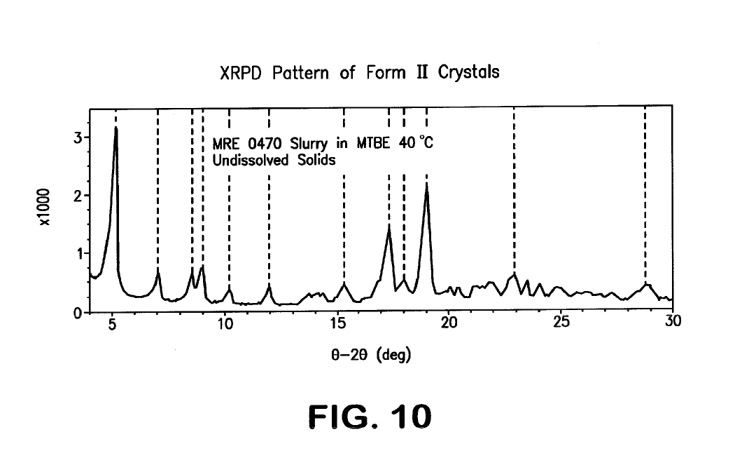

An example of an X-ray diffraction pattern exhibited by a Form III crystal form is substantially identical to that depicted in FIG. 10, having characteristic peaks, expressed in degrees 2-theta (2θ), of about 5.1±0.2, 7.1±0.2, 8.6±0.2, 9.0±0.2, 17.4±0.2 and 19.0±0.2. The present invention also provides a Form III crystal form that exhibits a X-ray diffraction pattern substantially the same as that depicted in FIG. 10, having characteristic diffraction peaks expressed in degrees 2-theta, and relative intensities (I/I₁) of approximately the values shown in Table 4 herein below:

TABLE 4
Form III crystals of binodenoson

| Angle (deg 2θ) | Relative intensity (I/I₁) |
|---|---|
| 5.1 ± 0.2 | 100 |
| 7.1 ± 0.2 | 21 |
| 8.6 ± 0.2 | 21 |
| 9.0 ± 0.2 | 23 |
| 10.2 ± 0.2 | 11 |
| 12.0 ± 0.2 | 13 |
| 15.3 ± 0.2 | 15 |
| 17.4 ± 0.2 | 45 |
| 18.0 ± 0.2 | 16 |
| 19.0 ± 0.2 | 67 |
| 23.0 ± 0.2 | 19 |
| 23.5 ± 0.2 | 17 |
| 24.1 ± 0.2 | 14 |

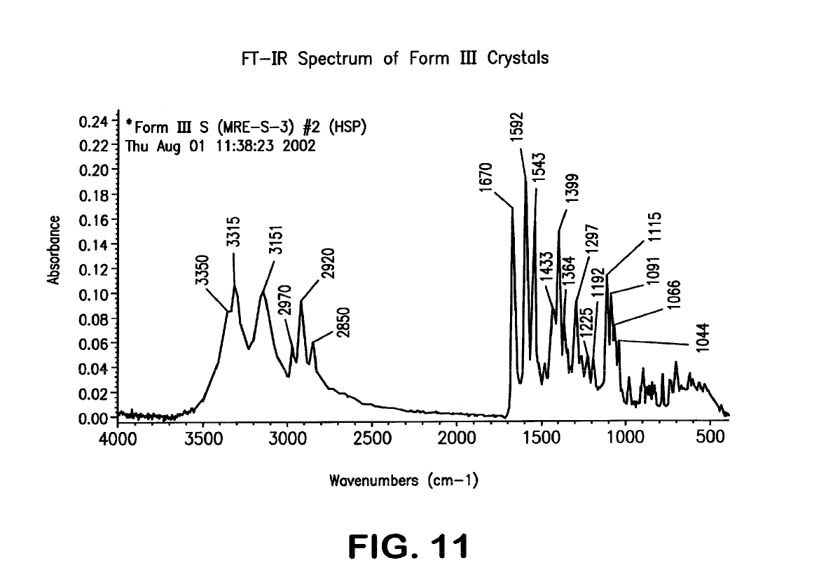

An example of an infrared reflectance spectrum of a Form III crystal form obtained by the diffuse reflectance method is shown in FIG. 11, and is characterized by reflectance bands at about 1669±2 and 1592±2 cm⁻¹.

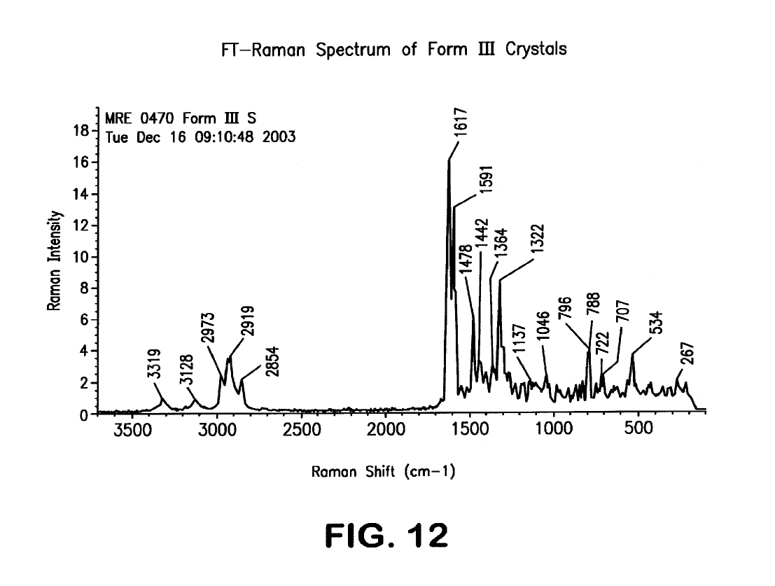

An example of a FT-Raman spectrum of a Form III crystal form obtained by the method described herein above is shown in FIG. 12, and is characterized by Raman shifts at about 1617±2 and 1591±2 cm⁻¹.

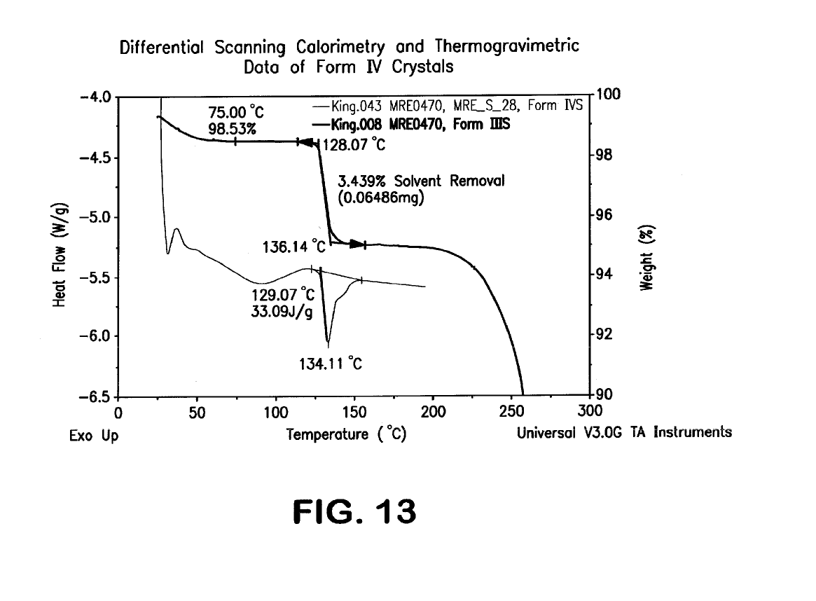

In yet another aspect, the present invention provides a crystal form of binodenoson, designated herein as Form IV, that is characterized by thermal DSC data, as measured by the DSC method described herein above, substantially identical to those depicted in FIG. 13.

Form IV crystals may be obtained, e.g., by slurry conversion of Form I crystals (anhydrous) in multiple solvent mixtures containing toluene (PhMe) and diisopropyl ether (i-Pr₂O), e.g., in a 75:25 mixture of PhMe and i-Pr₂O at 40-60° C.

Thermal DSC data of Form IV crystals of binodenoson exhibit a single endotherm with an extrapolated onset melting temperature in the range of about 129° C. to about 133° C. when heated at 10° C./min.

TGA of Form IV crystal form shows a characteristic weight loss of approximately 3.5% between 110° C. and 155° C. corresponding to loss of solvated i-Pr₂O. The ¹H-NMR spectrum of the Form IV crystals confirms the presence of i-Pr₂O. The Form IV crystal form appears to be a i-Pr₂O solvate.

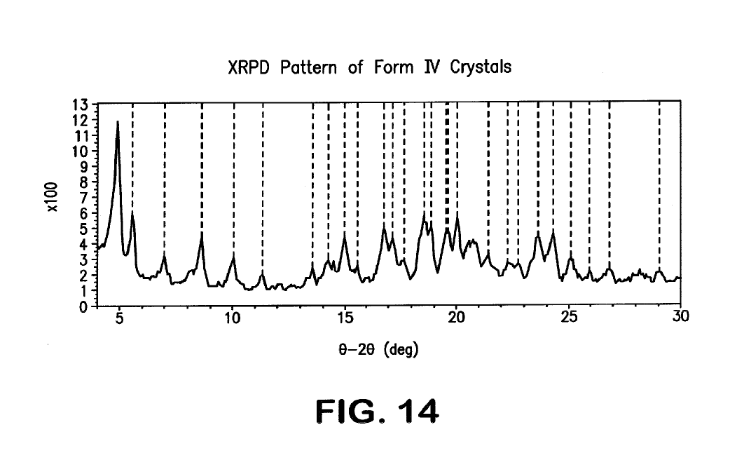

An example of an X-ray diffraction pattern exhibited by a Form IV crystal form is substantially identical to that depicted in FIG. 14, having characteristic peaks, expressed in degrees 2-theta (28), of about 4.9±0.2, 5.6±0.2, 8.6±0.2, 15.0±0.2, 16.8±0.2, 18.6±0.2, 18.9±0.2, 20.1±0.2, 23.7±0.2 and 24.3±0.2. The present invention also provides a Form IV crystal form that exhibits a X-ray diffraction pattern substantially the same as that depicted in FIG. 14, having characteristic diffraction peaks expressed in degrees 2-theta, and relative intensities (I/I₁) of approximately the values shown in Table 5 herein below:

TABLE 5
Form IV crystals of binodenoson

| Angle (deg 2θ) | Relative intensity (I/I₁) |
|---|---|
| 4.9 ± 0.2 | 100 |
| 5.6 ± 0.2 | 49 |
| 7.0 ± 0.2 | 28 |
| 8.6 ± 0.2 | 39 |
| 10.0 ± 0.2 | 26 |
| 15.0 ± 0.2 | 37 |
| 16.8 ± 0.2 | 41 |
| 17.2 ± 0.2 | 36 |
| 18.6 ± 0.2 | 47 |
| 18.9 ± 0.2 | 43 |
| 19.6 ± 0.2 | 39 |
| 19.7 ± 0.2 | 40 |
| 20.1 ± 0.2 | 47 |
| 21.5 ± 0.2 | 27 |
| 23.7 ± 0.2 | 37 |
| 24.3 ± 0.2 | 38 |

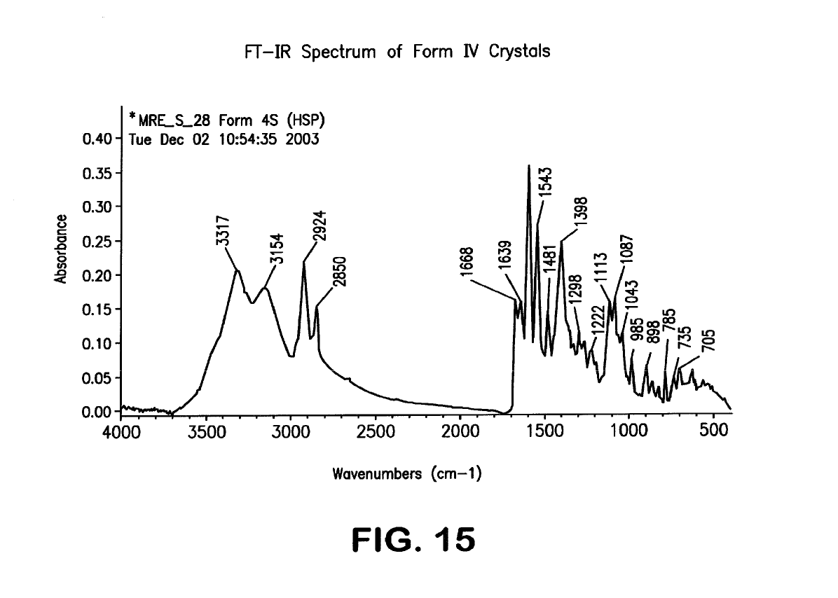

An example of an infrared reflectance spectrum of a Form IV crystal form obtained by the diffuse reflectance method is shown in FIG. 15, and is characterized by reflectance bands at about 1668±2, 1639±2 and 1591±2 cm⁻¹.

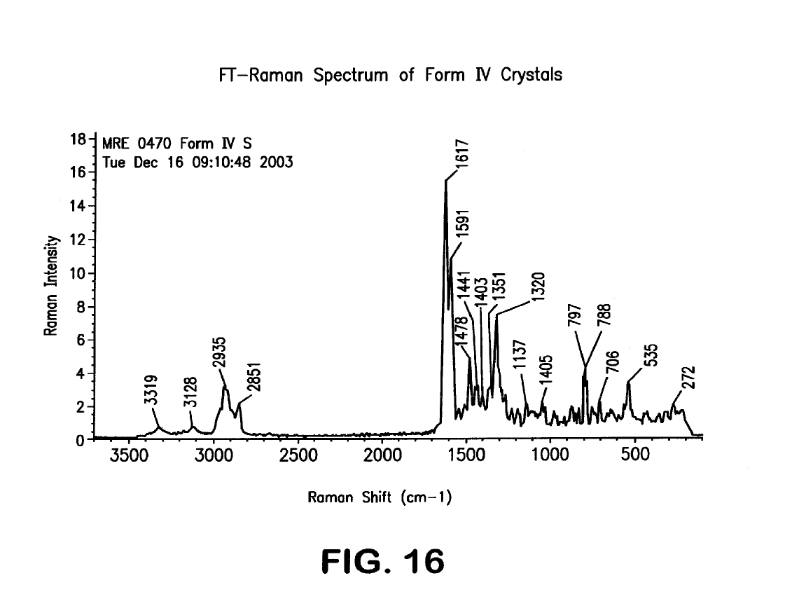

An example of a FT-Raman spectrum of a Form IV crystal form obtained by the method described herein above is shown in FIG. 16, and is characterized by Raman shifts at about 1617±2 and 1591±2 cm⁻¹.

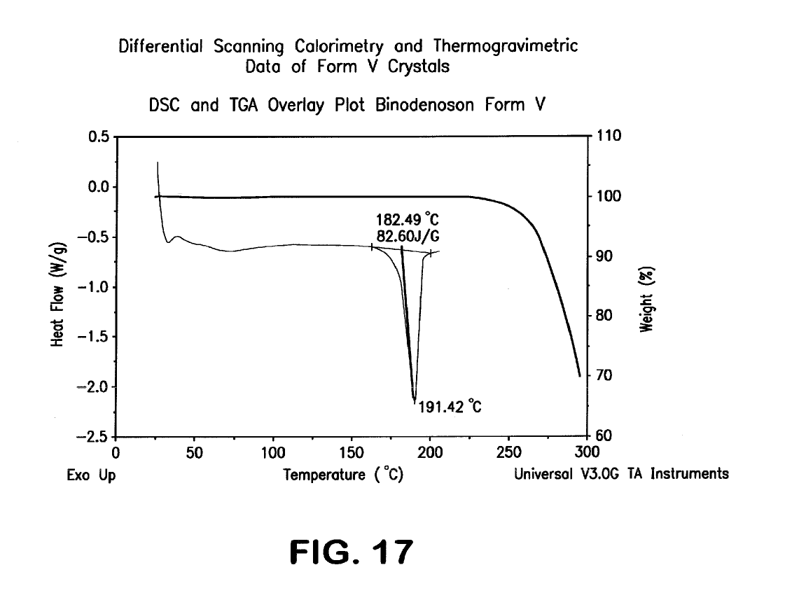

In yet another aspect, the present invention provides a crystal form of binodenoson, designated herein as Form V, that is characterized by thermal DSC data, as measured by the DSC method described herein above, substantially identical to those depicted in FIG. 17.

Form V crystals may be obtained, e.g., by slurry conversion of Form I crystals in a 90:10 mixture of PhMe and MeOH at 60° C.

Thermal DSC data of Form V crystals of binodenoson exhibit a single endotherm with an extrapolated onset melting temperature in the range of about 178° C. to about 183° C. when heated at 10° C./min.

TG analysis of the Form V crystals shows no significant weight loss between 25° C. and 225° C. The Form V crystal form appears to be an anhydrous crystal form of binodenoson.

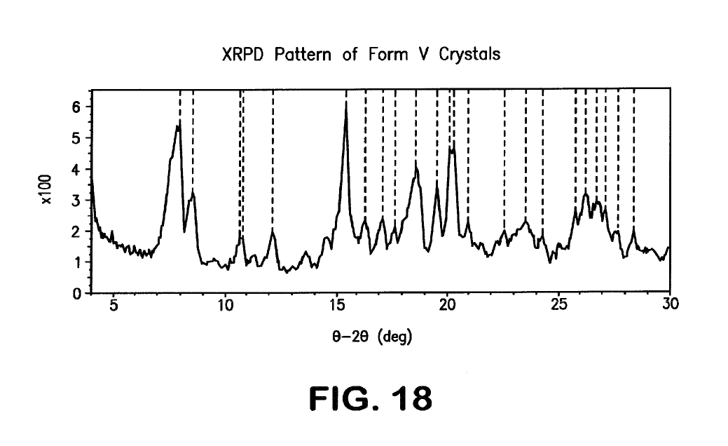

An example of an X-ray diffraction pattern exhibited by a Form V crystal form is substantially identical to that depicted in FIG. 18, having characteristic peaks, expressed in degrees 2-theta (2θ), of about 8.0±0.2, 8.5±0.2, 10.8±0.2, 12.1±0.2, 15.4±0.2, 17.1±0.2, 18.6±0.2, 19.6±0.2 and 20.3±0.2. The present invention also provides a Form V crystal form that exhibits a X-ray diffraction pattern substantially the same as that depicted in FIG. 18, having characteristic diffraction peaks expressed in degrees 2-theta, and relative intensities (I/I₁) of approximately the values shown in Table 6 herein below:

TABLE 6
Form V crystals of binodenoson

| Angle (deg 2θ) | Relative intensity (I/I₁) |
|---|---|
| 7.9 ± 0.2 | 91 |
| 8.0 ± 0.2 | 92 |
| 8.5 ± 0.2 | 55 |
| 10.7 ± 0.2 | 30 |
| 10.8 ± 0.2 | 30 |
| 12.1 ± 0.2 | 32 |
| 14.6 ± 0.2 | 30 |
| 15.4 ± 0.2 | 100 |
| 16.3 ± 0.2 | 40 |
| 17.1 ± 0.2 | 40 |
| 17.7 ± 0.2 | 36 |
| 18.6 ± 0.2 | 68 |
| 19.6 ± 0.2 | 57 |
| 20.2 ± 0.2 | 78 |
| 20.3 ± 0.2 | 79 |
| 21.0 ± 0.2 | 38 |
| 26.2 ± 0.2 | 53 |

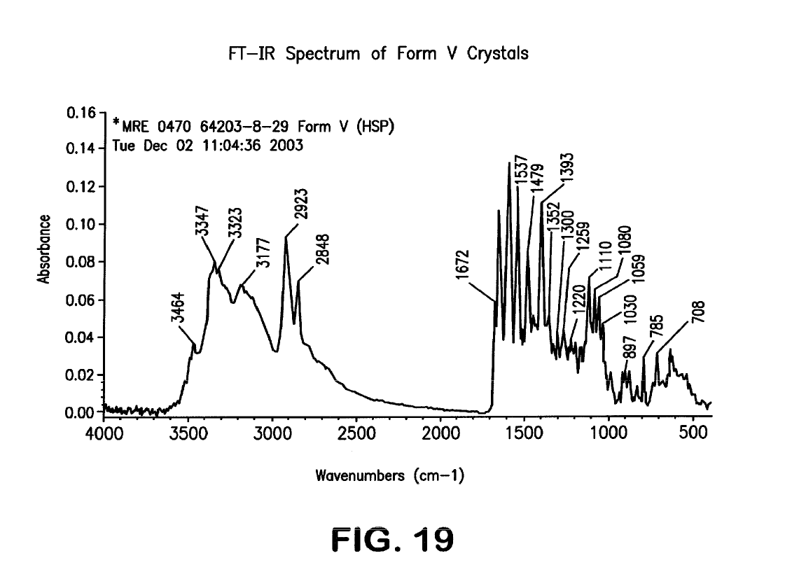

An example of an infrared reflectance spectrum of a Form V crystal form obtained by the diffuse reflectance method is shown in FIG. 19, and is characterized by reflectance bands at about 1672±2, 1650±2 and 1589±2 cm⁻¹.

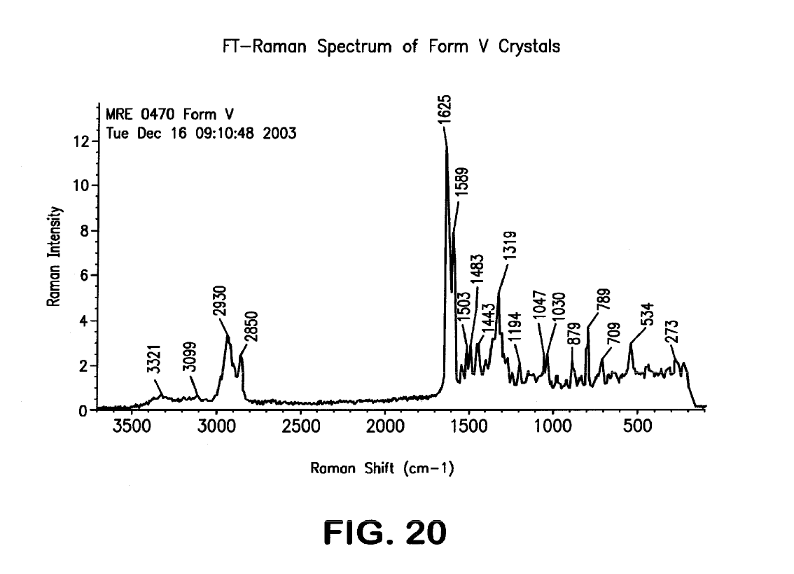

An example of a FT-Raman spectrum of a Form V crystal form obtained by the method described herein above is shown in FIG. 20, and is characterized by Raman shifts at about 1625±2 and 1589±2 cm⁻¹.

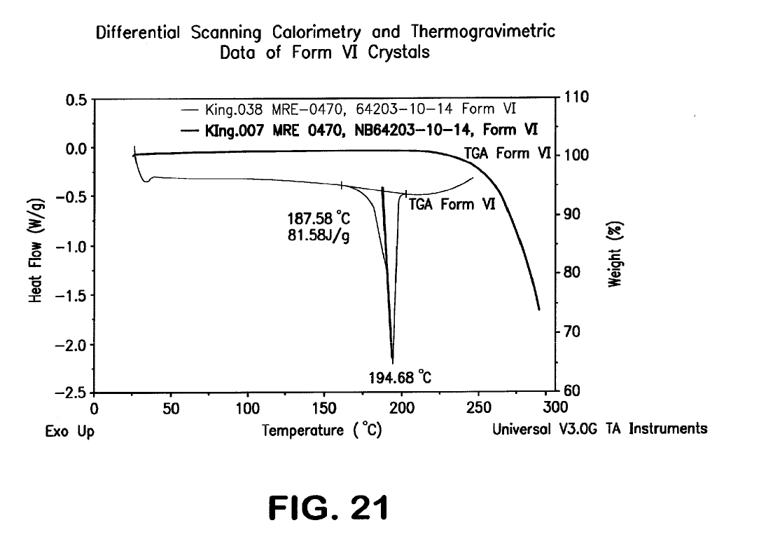

In yet another aspect, the present invention provides a crystal form of binodenoson, designated herein as Form VI, that is characterized by thermal DSC data, as measured by the DSC method described herein above, substantially identical to those depicted in FIG. 21.

Form VI crystals may be obtained, e.g., by slurry conversion of Form I crystals (anhydrous) in PhMe at 60° C.

Thermal DSC data of Form VI crystals of binodenoson exhibit a single endotherm with an extrapolated onset melting temperature in the range of about 183° C. to about 188° C. when heated at 10° C./min.

TG analysis of the Form VI crystals shows no significant weight loss between 25° C. and 225° C. The Form VI crystal form appears to be an anhydrous crystal form of binodenoson.

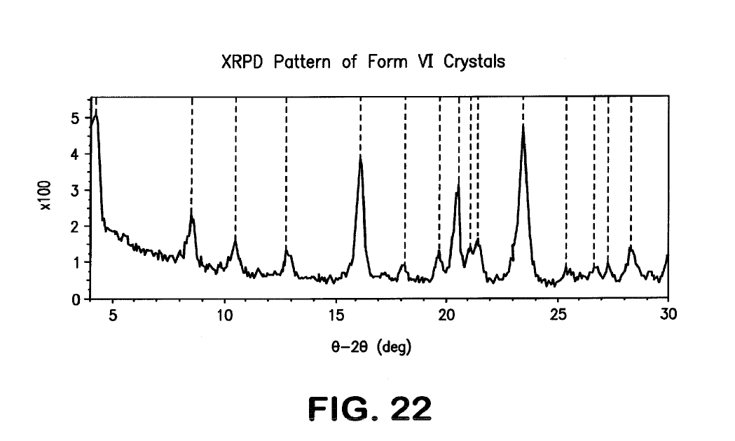

An example of an X-ray diffraction pattern exhibited by a Form VI crystal form is substantially identical to that depicted in FIG. 22, having characteristic peaks, expressed in degrees 2-theta (2θ), of about 4.2±0.2, 8.5±0.2, 10.5±0.2, 12.8±0.2, 16.1±0.2, 20.6±0.2 and 23.5±0.2. The present invention also provides a Form VI crystal form that exhibits a X-ray diffraction pattern substantially the same as that depicted in FIG. 22, having characteristic diffraction peaks expressed in degrees 2-theta, and relative intensities (I/I₁) of approximately the values shown in Table 7 herein below:

TABLE 7
Form VI crystals of binodenoson

| Angle (deg 2θ) | Relative intensity (I/I₁) |
|---|---|
| 4.2 ± 0.2 | 100 |
| 8.5 ± 0.2 | 47 |
| 10.5 ± 0.2 | 33 |
| 12.8 ± 0.2 | 26 |
| 16.1 ± 0.2 | 77 |
| 19.7 ± 0.2 | 26 |
| 20.6 ± 0.2 | 63 |
| 21.6 ± 0.2 | 29 |
| 23.5 ± 0.2 | 95 |
| 28.3 ± 0.2 | 27 |

An example of an infrared reflectance spectrum of a Form VI crystal form obtained by the diffuse reflectance method is shown in FIG. 19, and is characterized by reflectance bands at about 1647±2, 1595±2 and 1582±2 cm⁻¹.

An example of a FT-Raman spectrum of a Form VI crystal form obtained by the method described herein above is shown in FIG. 20, and is characterized by Raman shifts at about 1627±2 and 1595±2 cm⁻¹.

As described herein above, the present invention provides methods for the production of different crystal forms of binodenoson. For example, the present invention provides a method for the production of different crystal forms of binodenoson, wherein the method comprises forming a saturated solution of binodenoson in a suitable organic solvent, including mixed solvents, forming the crystals of binodenoson including hydrates and solvates, e.g., a monohydrate and a MTBE hemi-solvate of binodenoson, while evaporating the solution to dryness via isothermal evaporation, and characterizing the crystal form of binodenoson, e.g., Form I, Form II and Form III crystal forms of binodenoson.

Suitable solvents include, but are not limited to, lower alcohols such as MeOH, EtOH, 1-propanol and isopropanol (IPA), acetonitrile (ACN), dichloromethane (DCM), PhMe, ethers such as i-Pr₂O and MTBE, and esters such as ethyl acetate (EtOAc) and isopropyl acetate (i-PrOAc), and mixtures of solvents thereof, e.g., mixtures of EtOH and IPA, mixtures of 1-propanol and IPA, mixtures of IPA and MTBE, mixtures of i-Pr₂O and PhMe, mixtures EtOAc and DCM, mixtures of MTBE and EtOH, and mixtures of EtOAC and ACN.

The dissolution and crystallization may be carried out in several ways as will be apparent to those of ordinary skill in the art. For example, saturated solutions of binodenoson may be prepared by agitating excess of binodenoson, e.g., Form I crystals of binodenoson, in various solvent systems at an appropriate saturation temperature, e.g., a temperature ranging from about 25° C. to about 45° C. The mother liquor may then be separated from the residual solids, e.g., by pipetting or filtration. The mother liquor may optionally be heated at a temperature above the saturation temperature, e.g., a temperature ranging from about 5° C. to about 15° C. above the saturation temperature, to dissolve any remaining solids. The temperature of the solutions is then adjusted to the growth temperature, e.g., a temperature ranging from about 25° C. to about 60° C., and a nitrogen shear flow is introduced to begin solvent evaporation.

For example, a saturated solution of binodenoson may be prepared by agitating excess of Form I crystals in MTBE at 35° C. The mother liquor is separated from the residual solids by pipetting, then heated at 50° C. until all of the remaining solids are dissolved. The temperature is then adjusted back to 35° C., and a nitrogen shear flow (15 Fh) is introduced to begin solvent evaporation. The precipitated solids are characterized as Form III crystals of binodenoson.

In an alternative aspect of the present invention, solid forms of binodenoson may be suspended in a suitable solvent at a temperature of at least 10° C., preferably at a temperature ranging from about 25° C. to about 60° C. Under suitable conditions, a suspension/slurry results in which particles of solid are dispersed, and remain incompletely dissolved in the solvent. Preferably, the solids are maintained in a state of suspension by agitation, e.g., by shaking or stirring. The suspension/slurry is then kept at a temperature of 10° C. or higher, e.g., at a temperature ranging from about 25° C. to about 60° C., for a time sufficient to effect transformation of the starting solids into product crystals. The product crystals may then be isolated and dried using conventional methods in the art. In the presence of water, all crystal forms of binodenoson will convert to Form II hydrate.

Solvents suitable for use in this embodiment of the present invention include, but are not limited to, esters such as i-PrOAc and EtOAc, lower alcohols such as MeOH and EtOH, ethers such as MTBE and i-Pr₂O, and solvents such as DCM and PhMe, or a suitable mixture of solvents thereof, e.g., mixtures of i-Pr₂O and PhMe.

Two different types of slurry experiments may be performed, i.e., competitive and noncompetitive slurry experiments. Competitive slurry experiments are performed by mixing excess amounts of non-solvated polymorphic forms together in different solvents and agitating the mixture isothermally. These types of slurry experiments may be used to determine which form is more thermodynamically stable (under the conditions tested).

Noncompetitive slurry experiments are useful for identifying solvent mediated conditions useful for converting one crystalline form to another. In these experiments, excess material of a single crystal form is mixed with a solvent under isothermal conditions. These experiments rely on differences in solubility of the different polymorphic forms. As such, only modifications having a lower solubility (more stable) than the initial crystalline form can result from a noncompetitive slurry experiment. Essentially, when a polymorph is suspended in a suitable solvent, a saturated solution phase (eventually) results. The solution phase is saturated with respect to the polymorphic form dissolved. However, the solution is supersaturated with respect to any polymorphic form which is more stable (more stable forms have lower solubilities) than the polymorphic form initially dissolved. Therefore, any of the more stable forms can nucleate and precipitate from the solution phase.

Because the formation of nuclei of all given polymorphic forms of a given compound occur at different rates, anyone of the more stable polymorphic forms can result from a noncompetitive slurry experiment. Although, the solution phase exhibits the highest supersaturation with respect to the most stable form (with lowest solubility), the most stable polymorphic form is not always the form that nucleates first. The results of these experiments often depend on nucleation kinetics and the presence of impurities that may inhibit nuclei growth of other stable crystalline modifications.

When competitive slurry experiments are performed, the nucleation step is generally bypassed. Because the two (or more) forms placed into contact with the solvent have different solubilities, particles of the form with the lower solubility grow at the expense of the more soluble form. This occurs since the more soluble form is saturated, and the less soluble form is supersaturated. Sometimes (depending on the duration of the experiment) a competitive slurry experiment will result in a form which is more stable than either of the polymorphic forms initially placed into contact with the slurry solvent. This can occur, e.g., when the induction period for nucleation of a more stable form of the system has been exceeded. After nucleation of this third (or more stable) form, all residual solids in the slurry can be converted to the nucleated form via solvent mediated phase transition.

In general, slurry experiments are performed by agitating approximately 0.01 g to 2.5 g of material in 0.5 mL to 50 mL of slurry solvent. Uniform agitation and temperature control are accomplished using, e.g., Reacti-Therm heating modules and small Teflon coated stir bars. The duration of the slurry experiments is often around 24 h (although in some cases the experiments may be continued for several weeks). At the end of the slurry experiment, remaining undissolved solids are collected by vacuum filtration. To avoid inducing any type of physical change, the solids are not subjected to additional drying before the XRPD analysis. Illustrative examples of slurry experiments are summarized in Table 8 below:

TABLE 8

| Experi- | | | | | | |
|---|---|---|---|---|---|---|
| ment | | Initial | | Temp | | Final Form |
| Type | No. | Form(s) | Solvent | (° C.) | Time | (XRPD) |
| Non- | 1 | Form I | EtOAc | 40 | 4 h | amorphous |
| competi- | 2 | Form I | EtOH | 40 | 4 h | all dissolved |
| tive | 3 | Form I | MTBE | 40 | 4 h | III |
| | 4 | Form I | i-Pr₂O | 40 | 4 h | amorphous |
| | 5 | Form I | MeOH | 40 | 4 h | all dissolved |
| | 6 | Form I | DCM | 40 | 4 h | V |
| | 7 | Form I | EtOAc | 60 | 4 h | II^{b} |
| | 8 | Form I^{a} | PhMe: | 40 | >1 | IV |
| | | | i-Pr₂O | | day | |
| | | | 75:25 | | | |
| | 9 | Form I^{a} | PhMe: | 60 | >1 | V |
| | | | MeOH | | day | |
| | | | 90:10 | | | |
| | 10 | Form I^{a} | PhMe | 60 | >1 | VI |
| | | | | | day | |
| Competi- | 11 | Form I & | DCM | 25 | 24 h | II^{b} |
| tive | | II | | | | |
| | 12 | Form I & | i-PrOAc | 25 | 24 h | II^{b} |
| | | II | | | | |
| | 13 | Form I & | EtOAc | 25 | 24 h | II^{b} |
| | | II | | | | |

^{a}anhydrous; ^{b}monohydrate.

The data in Table 8 indicate that the non-competitive slurry experiments (experiments No. 1-7) produce a variety of results. Typically, if a non-competitive slurry experiment produces a solid-state change, the rate of transition is primarily a function of solubility, temperature and solvent identity. Other factors, such as impurity profile, hydrodynamics, etc. can also play a role.

In the current set of results, Form I crystals readily transforms into Form II crystals in EtOAc at 60° C. for 4 h (experiment No. 7). This would generally indicate that Form II is thermodynamically more stable than Form I. However, when the actual solubilities of the anhydrous Forms I and II are compared, e.g., in EtOH, Form II is found to have a much higher solubility. Therefore, it is deduced that the presence of residual water allows Form II to form an isostructural hydrate with a lower solubility than that of Form I (and lower solubility than anhydrous Form II). Thus, Form I appears to convert to Form II, but really converts to Form II hydrate. Because the solubility of anhydrous Form II is higher than that of Form I, Form I is thermodynamically more stable than Form II. During the solubility studies in dry EtOH, Form II is observed to completely dissolve and recrystallize as Form I. This demonstrates that when water is not present, Form I is less soluble and thermodynamically more stable than Form II. Thus, in the presence of residual water, Form II is obtained in the isostructural hydrate form, whereas in a completely anhydrous system, Form I results.

At 40° C. (experiments No. 1-6), many different results are observed. Experiment No. 3 affords Form III crystals. In experiments No. 2 and No. 5, all the solids dissolve and, therefore, these two solutions are allowed to evaporate slowly at room temperature. After 1-2 week(s), experiment No. 2 produce amorphous material and experiment No. 5 affords Form II crystals. Experiments No. 8, 9, and 10 produce Form IV, Form V and Form VI crystals, respectively.

Slurry experiments No. 1, No. 4, and No. 6 produce mostly amorphous looking pattern with broad features, but do exhibit some small X-ray scattering peaks. The small peaks observed in experiment No. 1 are attributed to Form II. The small peaks observed in experiment No. 4 are attributed to residual Form I. The amorphous looking pattern with broad features in experiments No. 1, No. 4, and No. 6 is attributed to Form V. Form V has been observed to develop slowly in many experiments as shown in FIG. 29. It is believed that the small particle size of Form V crystals often causes the XRPD pattern to appear amorphous with broad features. As time progresses, it is believed that particle ripening is responsible for the increase in quality of the XRPD pattern.

The conversion of Form I crystals to Form V in experiment No. 6 indicates that form V is more stable than Form I (at 40° C.) since only less soluble forms can nucleate during a slurry experiment. In experiments 4 and 6, Form V appears to have formed directly from Form I without the appearance of Form II.

In addition to the pure solvent noncompetitive slurry experiments performed, three competitive slurry experiments in Table 8 are performed at room temperature. These slurry experiments are composed of an approximately 50/50 mixture of Form I and Form II solids. Three different solvents are used to perform the competitive slurry experiments: DCM, i-PrOAc and EtOAc. As the results indicate, in each experiment Form I appears to completely convert to Form II crystals. While this would normally indicate that Form II is more thermodynamically stable than Form I, this result is believed to be a consequence of the formation of a low solubility hydrate of Form II rather than the solvent mediated polymorphic transformation of Form I into Form II.

As described herein above, the crystal forms of binodenoson, in particular Form II crystal form of binodenoson, preferably the hydrate thereof, may be employed for the manufacture of a pharmaceutical composition comprising an effective amount of binodenoson for the use of binodenoson in a subject, in need thereof, as a pharmacological stress agent to produce coronary vasodilation. The crystal forms of the present invention are especially useful for the manufacture of pharmaceutical compositions for achieving coronary vasodilation in subjects who cannot exercise adequately.

The term “effective amount” as used herein refers to an amount of crystals of binodenoson to be employed which is effective to provide coronary artery dilation (vasodilation).

The terms “subject or patient” are used interchangeably herein and include, but are not limited to, humans, dogs, cats, horses, pigs, cows, monkeys, and laboratory animals. The preferred subjects are humans.

The crystal forms of binodenoson may be formulated as pharmaceutical compositions and administered to a subject, such as a human patient, in a variety of forms adapted to the chosen route of administration, preferably parenterally, by intravenous, intramuscular, topical or subcutaneous routes.

Preferably, binodenoson is administered intravenously or intraperitoneally by infusion or injection. Solutions of binodenoson in water, may optionally be mixed with a nontoxic surfactant. Dispersions may also be prepared in glycerol, liquid polyethylene glycols, triacetin, and mixtures thereof, and in oils. Under ordinary conditions of storage and use, these preparations may contain a preservative to prevent the growth of microorganisms.

The pharmaceutical dosage forms suitable for injection or infusion can include sterile aqueous solutions or dispersions or sterile powders comprising binodenoson which are adapted for the extemporaneous preparation of sterile injectable or infusible solutions or dispersions, optionally encapsulated in liposomes. In all cases, the ultimate dosage form must be sterile, fluid and stable under the conditions of manufacture and storage.

The liquid carrier or vehicle can be a solvent or liquid dispersion medium comprising, e.g., water, ethanol, a polyol (e.g., glycerol, propylene glycol, liquid polyethylene glycols, and the like), vegetable oils, nontoxic glyceryl esters, and suitable mixtures thereof. The proper fluidity can be maintained, e.g., by the formation of liposomes, by the maintenance of the required particle size in the case of dispersions or by the use of surfactants. The prevention of the action of microorganisms can be brought about by various antibacterial and antifungal agents, e.g., parabens, chlorobutanol, phenol, sorbic acid, thimerosal, and the like. In many cases, it will be preferable to include isotonic agents, e.g., sugars, buffers and sodium chloride. Prolonged reflectance of the injectable compositions can be achieved by the use of agents delaying absorption, e.g., aluminum monostearate and gelatin.

Sterile injectable solutions are prepared by incorporating binodenoson, in any of its crystal forms, in an effective amount in the appropriate solvent with various of the other ingredients enumerated herein above (carrier), as required, followed by filter sterilization. In the case of sterile powders for the preparation of sterile injectable solutions, the preferred methods of preparation are vacuum drying and the freeze drying techniques, which yield a powder of binodenoson, in any of its forms, plus any additional desired ingredient present in the previously sterile-filtered solutions.

The crystal forms of binodenoson and compositions prepared by employing such crystal forms are administered as pharmacological stressors and used in conjunction with any one of several noninvasive diagnostic procedures to measure aspects of myocardial perfusion. For example, intravenous adenosine may be used in conjunction with thallium-201 myocardial perfusion imaging to assess the severity of myocardial ischemia. In this case, any one of several different radiopharmaceuticals may be substituted for thallium-201, such as those agents comprising technetium-99m, iodine-123, nitrogen-13, rubidium-82 and oxygen-13. Such agents include technetium-99m labeled radiopharmaceuticals, i.e., technetium-99m-sestamibi, technetium-99m-teboroxime, tetrafosmin and NOET, and iodine-123 labeled radiopharmaceuticals such as I-123-IPPA or BMIPP. Similarly, binodenoson may be administered as a pharmacological stressor in conjunction with radionuclide ventriculography to assess the severity of myocardial contractile dysfunction. In this case, radionuclide ventriculographic studies may be first pass or gated equilibrium studies of the right and/or left ventricle. Likewise, binodenoson may be administered as a pharmacological stressor in conjunction with echocardiography to assess the presence of regional wall motion abnormalities. Similarly, binodenoson may be administered as a pharmacological stressor in conjunction with invasive measurements of coronary blood flow such as by intracardiac catheter to assess the functional significance of stenotic coronary vessels.

Accordingly, the present invention provides a method of producing coronary vasodilation in a subject, in need thereof, comprising:
(a) incorporating an effective amount of a crystal form of binodenoson in an aqueous carrier suitable for parenteral administration to form a pharmaceutical composition;
(b) if required, reconstituting the pharmaceutical composition to form a pharmaceutical composition suitable for parenteral administration; and
(c) administering the pharmaceutical composition to the subject to produce coronary vasodilation.

Likewise, the present invention provides a method of assessing a coronary artery disease in a subject, in need thereof, comprising:
(a) incorporating an effective amount of a crystal form of binodenoson in an aqueous carrier suitable for parenteral administration to form a pharmaceutical composition;
(b) if required, reconstituting the pharmaceutical composition to form a pharmaceutical composition suitable for parenteral administration;
(c) administering the pharmaceutical composition to the subject to produce coronary vasodilation; and
(d) detecting a coronary artery disease in the subject.

The methods of the present invention typically involve the administration of binodenoson by intravenous infusion in doses which are effective to provide coronary artery dilation. Such effective doses may range from about 0.001 to about 20 μg/kg/min. Preferably, from about 0.01 to about 15 μg/kg/min of binodenoson is infused, more preferably from about 0.1 to about 10 μg/kg/min. Alternatively, binodenoson may be administered by a bolus administration, e.g., 1.5 μg/kg in 30 sec.

Preferred methods comprise the use of binodenoson as a substitute for exercise in conjunction with myocardial perfusion imaging to detect the presence and/or assess the severity of coronary artery disease in humans, wherein myocardial perfusion imaging is performed by any one of several techniques including radiopharmaceutical myocardial perfusion imaging using planar scintigraphy or single photon emission computed tomography (SPECT), positron emission tomograph (PET), nuclear magnetic resonance (NMR) imaging, perfusion contrast echocardiography, digital subtraction angiography (DSA) or ultrafast X-ray computed tomography (CINE CT).

A method is also provided comprising the use of binodenoson as a substitute for exercise in conjunction with imaging to detect the presence and/or assess the severity of ischemic ventricular dysfunction in humans wherein ischemic ventricular dysfunction is measured by any one of several imaging techniques including echocardiography, contrast ventriculography, or radionuclide ventriculography.

A method is also provided comprising the use of binodenoson as a coronary hyperemic agent in conjunction with means for measuring coronary blood flow velocity to assess the vasodilatory capacity (reserve capacity) of coronary arteries in humans, wherein coronary blood flow velocity is measured by any one of several techniques including Doppler flow catheter or digital subtraction angiography.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative of certain aspects of the present invention and are not a limitation of the scope of the present invention in any way.

### Example 1

### Preparation of Binodenoson Crystal Form I

A 12-liter, 3-neck round-bottom flask, equipped with an overhead stirrer, reflux condenser, pressure-equalizing addition funnel, thermometer, and gas inlet is purged with nitrogen. To the flask is added 2-hydrazinoadenosine (312 g), SDA-3C (denatured ethanol, 6.2 L) and water (0.62 L). The mixture is heated to 55±5° C. under a nitrogen atmosphere until a homogeneous solution is obtained. Cyclohexanecarboxaldehyde (0.143 L) is added to the mixture, which is then heated to reflux for a minimum of 30 min. Once less than 0.5% of the initial 2-hydrazinoadenosine is remaining, as determined by HPLC, heating is removed, and the mixture is concentrated to a foamy solid by rotary evaporation, followed by additional drying under high vacuum for at least 2 h. The crude product is dissolved in SDA-3C (1.8 L), then decolorizing carbon (27 g) is added. The mixture is stirred for 15 to 30 min at ambient temperature, filtered through a ceramic funnel fitted with a GF filter, and transferred to a clean 22-liter flask equipped with an overhead stirrer and pressure-equalizing addition funnel.

To the above SDA-3C solution of binodenoson is added MTBE (13.5 L) dropwise over approximately 2 h, with vigorous stirring at 15 to 25° C. After completion of the addition, the mixture is stirred an additional 50 to 90 min. The precipitated product is collected by filtration and washed with additional MTBE (2.25 L). The damp solid is transferred to a 20-liter rotary evaporator flask, to which is added ethanol USP (2.5 L). The ethanol is removed by evaporation on a rotary evaporator, keeping the bath temperature below 45° C. Additional ethanol USP (2.5 L) is added and evaporated a second time. The product is transferred to drying trays and placed in a vacuum drying oven at 55±5° C. for at least 15 h to afford binodenoson drug substance in crystal form I, as determined by powder X-ray diffraction.

### Example 2

### Preparation of Binodenoson Crystal Form II

2-Hydrazinoadenosine (up to 306.2 g) is charged into a 12 liter reaction flask equipped with mechanical stirrer, bearing, stir shaft, paddle, condenser, thermocouple, gas inlet, and bubbler. EtOH (20 mL/g of 2-hydrazinoadenosine used) and WFI (Water for Injection, 2 mL/g of 2-hydrazinoadenosine used) are added to the reaction flask. The solution is then sparged with UHP nitrogen for 15 min, then maintained under a nitrogen atmosphere while the mixture is heated to about 50 to 60° C. Cyclohexanecarboxaldehyde (1.12 equivalents, relative to 2-hydrazinoadensine) is then added by cannula under positive nitrogen pressure to the reaction flask. The reaction mixture is heated to reflux for at least 30 min, monitoring by HPLC until the amount of 2-hydrazinoadenosine remaining is less than 0.7%. The reaction mixture is transferred to a rotary evaporator bulb and concentrated in vacuo to a foamy solid by rotary evaporation, maintaining the bath temperature at 40±5° C. The heat to the rotary evaporator bath is removed and the crude binodenoson is dried for at least 2 h under reduced pressure.

EtOH (5 mL/g of 2-hydrazinoadenosine used) is added to the crude binodenoson in the rotary evaporator bulb and the mixture is heated to dissolve the solids. WFI (10 mL/g of 2-hydrazinoadenosine used) is added to the rotary evaporator bulb and heating is continued until a homogenous solution is obtained. The solution is allowed to cool to ambient temperature overnight. The drug substance is collected by filtration, the cake is washed with about 400 mL of WFI, then air dried at ambient temperature for 2 h.

The drug substance is recrystallized a second time by transferring the cake to a rotary evaporator bulb, EtOH (5 mL/g of 2-hydrazinoadenosine used) is added, and the mixture is heated to dissolve the solids. The homogeneous solution is filtered through a coarse sintered-glass funnel into a rotary evaporator bulb. WFI (10 mL/g of 2-hydrazinoadenosine used) is added to the rotary evaporator bulb and heating is applied until a homogenous solution is obtained. The solution is allowed to cool to ambient temperature overnight. The product is collected by filtration, and the cake is washed with about 400 mL of WFI.

The collected solids are transferred to a drying pan, then placed in a vacuum oven for at least 12 h at reduced pressure and 55±5° C. After determining the net weight of the solids, the solids are again dried for a minimum of additional 12 h at reduced pressure and 55±5° C. The net weight of the solids is then determined again. The 12-hour drying cycles are repeated under reduced pressure at 55±5° C. until the change in mass is less than 0.5%, affording binodenoson drug substance in crystal form II, as determined by powder X-ray diffraction.

### Example 3

### Preparation of Binodenoson Crystal Form II

Binodenoson drug substance, as a 50:50 mixture (approximate) of crystal form I (Example 1) and crystal form II (Example 2) (5.0 g), is added to a 250 mL 3-neck round bottom flask, equipped with a magnetic stir bar, thermometer, reflux condenser, pressure-equalizing addition funnel, and gas inlet. After purging the flask with nitrogen, DCM (75 mL) is added to the flask through the addition funnel. The suspension is stirred at 25° C. for 24 h. The solid is collected by filtration, affording binodenoson crystal form II, as determined by powder X-ray diffraction.

### Example 4

### Preparation of Binodenoson Crystal Form II

Binodenoson drug substance, as a 50:50 mixture (approximate) of crystal form I (Example 1) and crystal form II (Example 2) (5.0 g), is added to a 250 mL 3-neck round bottom flask, equipped with a magnetic stir bar, thermometer, reflux condenser, pressure-equalizing addition funnel, and gas inlet. After purging the flask with nitrogen, EtOAc (75 mL) is added to the flask through the addition funnel. The suspension is stirred at 25° C. for 24 h. The solid is collected by filtration, affording binodenoson crystal form II, as determined by powder X-ray diffraction.

### Example 5

### Preparation of Binodenoson Crystal Form II

Binodenoson drug substance, as a 50:50 mixture (approximate) of crystal form I (Example 1) and crystal form II (Example 2) (5.0 g), is added to a 250 mL 3-neck round bottom flask, equipped with a magnetic stir bar, thermometer, reflux condenser, pressure-equalizing addition funnel, and gas inlet. After purging the flask with nitrogen, i-PrOAc (75 mL) is added to the flask through the addition funnel. The suspension is stirred at 25° C. for 24 h. The solid is collected by filtration, affording binodenoson crystal form II, as determined by powder X-ray diffraction.

### Example 6

### Preparation of Binodenoson Crystal Form II

Binodenoson drug substance, as crystal form I (Example 1) and crystal form II (Example 2) (5.0 g), is added to a 250 mL 3-neck round bottom flask, equipped with a magnetic stir bar, thermometer, reflux condenser, pressure-equalizing addition funnel, without protection from moisture. PhMe (75 mL) is added to the flask through the addition funnel, and the suspension is stirred at 60° C. for 14 days. The solid is collected by filtration, affording binodenoson crystal form II, as determined by powder X-ray diffraction.

### Example 7

### Preparation of Binodenoson Crystal Form III

Binodenoson drug substance, as crystal form I (5.0 g), is added to a 250 mL 3-neck round bottom flask, equipped with a magnetic stir bar, thermometer, reflux condenser, pressure-equalizing addition funnel, and gas inlet. After purging the flask with nitrogen, MTBE (75 mL) is added to the flask through the addition funnel. The suspension is gently warmed to 40° C. for 4 h, cooled to room temperature, and the solid collected by filtration, affording binodenoson crystal form III, as determined by powder X-ray diffraction.

### Example 8

### Preparation of Binodenoson Crystal Form IV

Binodenoson drug substance, as crystal form I (Example 1) (150 mg), is added to a 10 mL 2-neck round bottom flask, equipped with a magnetic stir bar, thermometer, reflux condenser, and gas inlet. A 1:3 mixture of i-Pr₂O and PhMe (5 mL) is added to the flask and the suspension is heated to 40° C. for 3 days, cooled to room temperature, and the solid is collected by filtration, affording binodenoson crystal form IV, as determined by powder X-ray diffraction.

### Example 9

### Preparation of Binodenoson Crystal Form V

Binodenoson drug substance, as crystal form I (Example 1) (2.5 g), is added to a 250 mL 3-neck round bottom flask, equipped with a magnetic stir bar, thermometer, reflux condenser, pressure-equalizing addition funnel, and gas inlet. After purging the flask with nitrogen, EtOAc (75 mL) is added to the flask through the addition funnel. The suspension is warmed to 60° C. with stirring for 15 days, cooled to room temperature, and the solid is collected by filtration, affording binodenoson crystal form V, as determined by powder X-ray diffraction.

### Example 10

### Preparation of Binodenoson Crystal Form V

Binodenoson drug substance, as a 50:50 mixture (approximate) of crystal form II (Example 2) and crystal form V (Example 8) (5.0 g), is added to a 250 mL 3-neck round bottom flask, equipped with a magnetic stir bar, thermometer, reflux condenser, pressure-equalizing addition funnel, and gas inlet. After purging the flask with nitrogen, anhydrous EtOAc (75 mL) is added to the flask through the addition funnel. The suspension is stirred at 25° C. for 2 weeks. The solid is collected by filtration, affording binodenoson crystal form V, as determined by powder X-ray diffraction.

### Example 11

### Preparation of Binodenoson Crystal Form VI

Binodenoson drug substance, as crystal form I (Example 1) (150 mg), is dried under vacuum at 105° C. for 35 min, then added to a 10 mL 2-neck round bottom flask, equipped with a magnetic stir bar, thermometer, reflux condenser, and gas inlet. After purging the flask with nitrogen, PhMe (5 mL) is added to the flask and the suspension is warmed to 60° C. with stirring for 10 days. After cooling to room temperature, the solid is collected by filtration, affording binodenoson crystal form VI, as determined by powder X-ray diffraction.

### Example 12

### Formulation of Binodenoson

WFI is charged into a suitable reaction vessel and sparged with nitrogen. Sodium phosphate dibasic, anhydrous, (1.080 g) is added to the WFI and mixed. Mannitol (1.320 g) is added to the reaction vessel and mixed with heating at approximately 60° C. (±5° C.). After the heating is discontinued, the temperature continues to rise. Once the solution reaches approximately 65° C. (±5° C.), it is mixed for at least 10 min. The solution is then cooled to approximately 20° C. (±2° C.) while mixing. The mixing is continued and the solution is sparged with nitrogen for at least 10 min. The mixing and sparging is continued as the pH of the bulk solution is adjusted between 9.8 to 10.2 by addition of 0.1 N sodium hydroxide. Alternatively, phosphoric acid may be used to adjust the solution if it is too basic (note that no batches manufactured thus far have required phosphoric acid adjustment). Following the pH adjustment, the solution is brought to volume (40.0 mL) using WFI and mixed for at least 15 min. If necessary, the pH may be adjusted again to between 9.8 to 10.2 using 0.1 N NaOH (or phosphoric acid).

A bulk solution of binodenoson is separately prepared by dissolving the required quantity of drug substance (0.010 g of crystal form II) in a minimum amount of MeOH (typically approximately 4.5 mL per L of bulk formulated drug product). This mixture may be sonicated or mixed until the binodenoson is dissolved, as determined by visual examination.

Under nitrogen overlay, the binodenoson bulk solution is transferred to the container holding the phosphate/mannitol buffer. The solution is mixed with cooling to approximately 5° C. (±3° C.).

The binodenoson bulk solution is filtered using a 0.2 μm filter into previously washed and depyrogenated vials. The filled vials are partially capped with sterilized siliconized stoppers, then lyophilized After removal from the lyophilization chamber, the vials are capped.

What is claimed is:

1. A crystal form of 2-{2-[(cyclohexyl)methylene]hydrazino}adenosine (binodenoson) which crystal form is substantially free of other polymorphic forms of binodenoson and has at least one of the following properties:
(a) an endotherm by differential scanning calorimetry with an extrapolated onset melting temperature in the range of about 139° C. to about 146° C. when heated at 10° C./min;
(b) a X-ray diffraction pattern with characteristic X-ray diffraction peaks at diffraction angles (2θ) of about 5.7±0.2, 10.2±0.2, 14.6±0.2, 19.9±0.2, 21.1±0.2 and 24.6±0.2;
(c) an infrared reflectance spectrum with reflectance bands at about 1668±2 and 1592±2; and
(d) a Raman spectrum with Raman shifts at about 1618±2 and 1593±2 cm⁻¹.

2. A crystal form according to claim 1, which crystal form has characteristic X-ray diffraction peaks at diffraction angles (2θ), and relative intensities (I/I₁) of about:

| Angle (deg 2θ) | Relative intensity (I/I₁) |
|---|---|
| 5.7 ± 0.2 | 100 |
| 10.2 ± 0.2 | 40 |
| 11.4 ± 0.2 | 22 |
| 14.4 ± 0.2 | 21 |
| 14.6 ± 0.2 | 25 |
| 15.6 ± 0.2 | 21 |
| 19.9 ± 0.2 | 38 |
| 20.5 ± 0.2 | 21 |
| 20.8 ± 0.2 | 17 |
| 21.1 ± 0.2 | 29 |
| 22.0 ± 0.2 | 17 |
| 24.2 ± 0.2 | 16 |
| 24.6 ± 0.2 | 27 |

3. A crystal form according to claim 1, which crystal form has all four of the properties (a), (b), (c) and (d).

4. A crystal form of binodenoson which crystal form is substantially free of other polymorphic forms of binodenoson and has at least one of the following properties:
(a) an endotherm by differential scanning calorimetry with an extrapolated onset melting temperature in the range of about 149° C. to about 154° C. when heated at 10° C./min;
(b) a X-ray diffraction pattern with characteristic X-ray diffraction peaks at diffraction angles (2θ) of about 5.5±0.2, 10.4±0.2, 16.8±0.2, 20.2±0.2 and 26.0±0.2;
(c) an infrared reflectance spectrum with reflectance bands at about 1646±2 and 1598±2 cm⁻¹; and
(d) a Raman spectrum with Raman shifts at about 1622±2 and 1588±2 cm⁻¹.

5. A crystal form according to claim 4, which crystal form has characteristic X-ray diffraction peaks at diffraction angles (2θ), and relative intensities of about:

| Angle (deg 2θ) | Relative intensity (I/I₁) |
|---|---|
| 5.5 ± 0.2 | 100 |
| 10.4 ± 0.2 | 15 |
| 16.8 ± 0.2 | 15 |
| 20.2 ± 0.2 | 18 |
| 26.0 ± 0.2 | 50 |

6. A crystal form according to claim 4, which crystal form has all four of the properties (a), (b), (c) and (d).

7. A crystal form of anhydrous binodenoson which crystal form is substantially free of other polymorphic forms of binodenoson and has at least one of the following properties:
(a) an endotherm by differential scanning calorimetry with an extrapolated onset melting temperature in the range of about 142° C. to about 145° C. when heated at 10° C./min;
(b) a X-ray diffraction pattern with characteristic X-ray diffraction peaks at diffraction angles (29) of about 5.1±0.2, 7.1±0.2, 8.6±0.2, 9.0±0.2, 17.4±0.2 and 19.0±0.2;
(c) an infrared reflectance spectrum with reflectance bands at about 1669±2 and 1592±2 cm⁻¹; and
(d) a Raman spectrum with Raman shifts at about 1617±2 and 1591±2 cm⁻¹.

8. A crystal form according to claim 7, which crystal form has characteristic X-ray diffraction peaks at diffraction angles (2θ), and relative intensities of about:

| Angle (deg 2θ) | Relative intensity (I/I₁) |
|---|---|
| 5.1 ± 0.2 | 100 |
| 7.1 ± 0.2 | 21 |
| 8.6 ± 0.2 | 21 |
| 9.0 ± 0.2 | 23 |
| 10.2 ± 0.2 | 11 |
| 12.0 ± 0.2 | 13 |
| 15.3 ± 0.2 | 15 |
| 17.4 ± 0.2 | 45 |
| 18.0 ± 0.2 | 16 |
| 19.0 ± 0.2 | 67 |
| 23.0 ± 0.2 | 19 |
| 23.5 ± 0.2 | 17 |
| 24.1 ± 0.2 | 14 |

9. A crystal form according to claim 7, which crystal form has all four of the properties (a), (b), (c) and (d).

10. A crystal form of anhydrous binodenoson which crystal form is substantially free of other polymorphic forms of binodenoson and has at least one of the following properties:
(a) an endotherm by differential scanning calorimetry with an extrapolated onset melting temperature in the range of about 129° C. to about 133° C. when heated at 10° C./min;
(b) a X-ray diffraction pattern with characteristic X-ray diffraction peaks at diffraction angles (2θ) of about 4.9±0.2, 5.6±0.2, 8.6±0.2, 15.0±0.2, 16.8±0.2, 18.6±0.2, 18.9±0.2, 20.1±0.2, 23.7±0.2 and 24.3±0.2;
(c) an infrared reflectance spectrum with reflectance bands at about 1668±2, 1639±2 and 1591±2 cm⁻¹; and
(d) a Raman spectrum with Raman shifts at about 1617±2 and 1591±2 cm⁻¹.

11. A crystal form according to claim 10, which crystal form has characteristic X-ray diffraction peaks at diffraction angles (2θ), and relative intensities of about:

| Angle (deg 2θ) | Relative intensity (I/I₁) |
|---|---|
| 4.9 ± 0.2 | 100 |
| 5.6 ± 0.2 | 49 |
| 7.0 ± 0.2 | 28 |
| 8.6 ± 0.2 | 39 |
| 10.0 ± 0.2 | 26 |
| 15.0 ± 0.2 | 37 |
| 16.8 ± 0.2 | 41 |
| 17.2 ± 0.2 | 36 |
| 18.6 ± 0.2 | 47 |
| 18.9 ± 0.2 | 43 |
| 19.6 ± 0.2 | 39 |
| 19.7 ± 0.2 | 40 |
| 20.1 ± 0.2 | 47 |
| 21.5 ± 0.2 | 27 |
| 23.7 ± 0.2 | 37 |
| 24.3 ± 0.2 | 38 |

12. A crystal form according to claim 10, which crystal form has all four of the properties (a), (b), (c) and (d).

13. A crystal form of anhydrous binodenoson which crystal form is substantially free of other polymorphic forms of binodenoson and has at least one of the following properties:
(a) an endotherm by differential scanning calorimetry with an extrapolated onset melting temperature in the range of about 178° C. to about 183° C. when heated at 10° C./min;
(b) a X-ray diffraction pattern with characteristic X-ray diffraction peaks at diffraction angles (2θ) of about 8.0±0.2, 8.5±0.2, 10.8±0.2, 12.1±0.2, 15.4±0.2, 17.1±0.2, 18.6±0.2, 19.6±0.2 and 20.3±0.2;
(c) an infrared reflectance spectrum with reflectance bands at about 1672±2, 1650±2 and 1589±2 cm⁻¹; and
(d) a Raman spectrum with Raman shifts at about 1625±2 and 1589±2 cm⁻¹.

14. A crystal form according to claim 13, which crystal form has characteristic X-ray diffraction peaks at diffraction angles (2θ), and relative intensities of about:

| Angle (deg 2θ) | Relative intensity (I/I₁) |
|---|---|
| 7.9 ± 0.2 | 91 |
| 8.0 ± 0.2 | 92 |
| 8.5 ± 0.2 | 55 |
| 10.7 ± 0.2 | 30 |
| 10.8 ± 0.2 | 30 |
| 12.1 ± 0.2 | 32 |
| 14.6 ± 0.2 | 30 |
| 15.4 ± 0.2 | 100 |
| 16.3 ± 0.2 | 40 |
| 17.1 ± 0.2 | 40 |
| 17.7 ± 0.2 | 36 |
| 18.6 ± 0.2 | 68 |
| 19.6 ± 0.2 | 57 |
| 20.2 ± 0.2 | 78 |
| 20.3 ± 0.2 | 79 |
| 21.0 ± 0.2 | 38 |
| 26.2 ± 0.2 | 53 |

15. A crystal form according to claim 13, which crystal form has all four of the properties (a), (b), (c) and (d).

16. A crystal form of anhydrous binodenoson which crystal form is substantially free of other polymorphic forms of binodenoson and has at least one of the following properties:
(a) an endotherm by differential scanning calorimetry with an extrapolated onset melting temperature in the range of about 183° C. to about 188° C. when heated at 10° C./min;
(b) a X-ray diffraction pattern with characteristic X-ray diffraction peaks at diffraction angles (2θ) of about 4.2±0.2, 8.5±0.2, 10.5±0.2, 12.8±0.2, 16.1±0.2, 20.6±0.2 and 23.5±0.2;
(c) an infrared reflectance spectrum with reflectance bands at about 1647±2, 1595±2 and 1582±2 cm⁻¹; and
(d) a Raman spectrum with Raman shifts at about 1627±2 and 1595±2 cm⁻¹.

17. A crystal form according to claim 16, which crystal form has characteristic X-ray diffraction peaks at diffraction angles (2θ), and relative intensities of about:

| Angle (deg 2θ) | Relative intensity (I/I₁) |
|---|---|
| 4.2 ± 0.2 | 100 |
| 8.5 ± 0.2 | 47 |
| 10.5 ± 0.2 | 33 |
| 12.8 ± 0.2 | 26 |
| 16.1 ± 0.2 | 77 |
| 19.7 ± 0.2 | 26 |
| 20.6 ± 0.2 | 63 |
| 21.6 ± 0.2 | 29 |
| 23.5 ± 0.2 | 95 |
| 28.3 ± 0.2 | 27 |

18. A crystal form according to claim 16, which crystal form has all four of the properties (a), (b), (c) and (d).

19. A method for the manufacture of a pharmaceutical composition by employing a crystal form according to any one of claims 1 to 18, for the use of binodenoson in a subject, in need thereof, as a pharmacological stress agent to produce coronary vasodilation.

20. A method of producing coronary vasodilation in a subject, in need thereof, comprising:
(a) incorporating an effective amount of a crystal form according to any one of claims 1 to 18 in an aqueous carrier suitable for parenteral administration to form a pharmaceutical composition;
(b) if required, reconstituting the pharmaceutical composition to form a pharmaceutical composition suitable for parenteral administration; and
(c) administering the pharmaceutical composition to the subject to produce coronary vasodilation.

21. A method of assessing a coronary artery disease in a subject, in need thereof, comprising:
(a) dissolving an effective amount of a crystal form according to any one of claims 1 to 18 in an aqueous solution suitable for parenteral administration to form a pharmaceutical composition;
(b) if required, reconstituting the pharmaceutical composition to form a pharmaceutical composition suitable for parenteral administration;
(c) administering the pharmaceutical composition to the subject to produce coronary vasodilation; and
(d) detecting a coronary artery disease in the subject.
